(19) United States
(12) Patent Application Publication
(10) Pub. No.: US 2011/0171736 A1
(43) Pub. Date: Jul. 14, 2011

(54) FLUORESCENT ISOTOPE TAGS AND THEIR METHOD OF USE

(75) Inventors: Brian AGNEW, Eugene, OR (US); Kyle Richard Gee, Springfield, OR (US)

(73) Assignee: LIFE TECHNOLOGIES CORPORATION, Carlsbad, CA (US)

(21) Appl. No.: 12/944,316

(22) Filed: Nov. 11, 2010

Related U.S. Application Data
(63) Continuation of application No. 12/487,945, filed on Jun. 19, 2009, now abandoned.
Application No. 12/487,945 is a continuation of application No. 12/177,057, filed on Jul. 21, 2008, now abandoned.
Application No. 12/177,057 is a continuation of application No. 11/157,467, filed on Jun. 20, 2005, now abandoned.
(60) Provisional application No. 60/580,842, filed on Jun. 18, 2004.

(51) Int. Cl. G01N 33/50; C07D 207/46; C07D 311/88; C07D 405/12; C07D 235/02

(52) U.S. Cl. 436/86; 548/542; 549/394; 544/375; 548/303.7

(57) ABSTRACT

The present invention provides novel reactive fluorescent compounds that incorporate stable isotopic (deuterium, 13-carbon, 15-nitrogen, 18-oxygen) substitutions. The invention includes the use of these compounds, in combination with non-isotopically substituted analogs, for the purification, identification and relative quantification of proteins, peptides, saccharides, metabolites, and other biologically important compounds by combining liquid chromatography (LC) and mass spectrometry (MS). Fluorescent labeling of target compounds in this manner provides orders-of-magnitude sensitivity enhancement over traditional stable isotope labels, and also affords the possibility of simultaneous multiplexed analysis due to the multiwavelength nature of different fluorophores.

### CROSS-REFERENCE TO RELATED APPLICATIONS

This application is a continuation of U.S. Ser. No. 12/487,945, filed Jun. 19, 2009, which is a continuation of U.S. Ser. No. 12/177,057, now abandoned, filed Jul. 21, 2008, which is a continuation of U.S. Ser. No. 11/157,467, now abandoned, filed Jun. 20, 2005, which claims priority to U.S. Ser. No. 60/580,842, filed Jun. 18, 2004, all of which disclosures are hereby incorporated herein by reference in their entirety.

### FIELD OF THE INVENTION

The present invention relates to novel fluorescent isotope tags for use in methods for identifying specific proteins in complex protein mixtures. In particular, the methods of the present invention relate to the rapid identification of differentially-expressed proteins from two different samples, e.g., different tissues, different cell types or different cell states, using liquid chromatography (LC) and mass spectrometry (MS). The invention has applications in the fields of cell biology, neurology, nutrition, immunology, cancer, infectious diseases and proteomics.

### BACKGROUND OF THE INVENTION

Proteomics research aims to study the expression levels and function of proteins, and subsets thereof, present in biological samples. Quantitation of these expression levels is extremely difficult, in part because protein content in a cell is dynamic and cannot be easily amplified. Adding to the difficulty, there is a difference of several orders of magnitude between the most abundant and least abundant proteins. Historically, 2D-gel electrophoresis is used to separate mixtures of proteins, allowing for identification by excision of protein spots from the gels followed by characterization by Edman degradation or mass spectrometry (MS) techniques. Relative quantitation of proteins in healthy and diseased cells, for example, can be performed; however the approach is time consuming, and proteomic coverage is incomplete as many high molecular weight and basic proteins do not resolve well on 2-D gels.

The isotope coded affinity tag (ICAT) technique takes advantage of differential covalent labeling of cysteine residues in proteins with stable isotopically labeled (or not) reagents, followed by affinity and ion exchange chromatography (U.S. Pat. No. 6,670,194). Peptides in individual ion exchange fractions are then identified using online reversed phase liquid chromatography coupled to mass spectrometry. Relative amounts of peptides are determined by comparison of ion currents obtained from peptides labeled with heavy (deuterium, 15-nitrogen, 13-carbon) or light (hydrogen, 14-nitrogen, 12-carbon) mass tags in a mass spectrometer. This method allows for the relative measurement of cysteine-containing peptides from two related samples and offers the potential of reproducible, quantitative comparisons and relatively rapid identification of a number of cellular proteins when coupled with liquid chromatography and tandem mass spectrometry methods. However, this method suffers from lack of sensitivity because of the intrinsically low resolution of proteins and peptides during chromatography.

In another method for comparing the levels of cellular components, such as proteins, samples are incubated with light and heavy isotope reagents (U.S. Pat. No. 6,391,649). A first sample of biological matter, such as cells, is cultured in a first medium and a second sample of the same biological matter is cultured in a second medium, wherein at least one isotope in the second medium has a different abundance than the abundance of the same isotope in the first medium. One of the samples is modulated, such as by treatment with a bacteria, a virus, a drug, hormone, a chemical or an environmental stimulus. The samples are combined and at least one protein is removed. The removed protein is subjected to mass spectroscopy to develop a mass spectrum. A ratio is computed between the peak intensities of at least one closely spaced pair of peaks to determine the relative abundance of the protein in each sample. The protein is identified by the mass spectrum or through other techniques known in the art.

Fluorescent dyes are widely used as tracers for localization of biological structures by fluorescence microscopy, for quantification of analytes by fluorescence immunoassay, for flow cytometric analysis of cells, for measurement of physiological state of cells and other applications (Kanaoka, Angew. Chem. Intl. Ed. Engl. 16: 137 (1977); Hemmila, Clin. Chem. 31: 359 (1985)). Among the advantages of fluorescent agents over other types of absorption dyes include the detectability of emission at a wavelength distinct from the excitation, the orders of magnitude greater detectability of fluorescence emission over light absorption, the generally low level of fluorescence background in most biological samples and the measurable intrinsic spectral properties of fluorescence polarization (Jolley et al., Clin. Chem. 27: 1190 (1981)), lifetime (U.S. Pat. No. 4,374,120) and excited state energy transfer (U.S. Pat. Nos. 3,996,345; and 4,542,104).

Thus, tagging or covalently labeling of proteins and peptides with fluorescent molecules is a well established technique for quantifying and purifying labeled molecules. In this instance, the fluorescent label dramatically increases sensitivity of detection, allowing for very small quantities of labeled protein or peptide to be isolated from non-labeled components in a mixture and analyzed. The present invention involves combining for the first time differential labeling of protein samples with stable isotopically coded fluorescent compounds and fluorescent tags. The “heavy” (13-carbon, deuterium, and/or 15-nitrogen) and “light” (12-carbon, hydrogen, and/or 14-nitrogen) fluorescent tagging reagents have identical or near-identical chromatographic properties upon binding proteins or peptides. Thus, the present method provides an improvement over currently used ICAT methods by providing a means for sensitive detection of differentially labeled proteins or peptides that may or may not require affinity separation prior to analysis.

The present invention overcomes the limitations of isotope coded affinity tags for the selective identification of proteins by providing fluorescent isotope tags and a method of using the tags with a modified ICAT methodology. In addition the present invention eliminates the need to incubate sample with stable isotopes for incorporation into expressed proteins.

### SUMMARY OF THE INVENTION

This invention provides analytical reagents and mass spectrometry-based methods using these reagents for the rapid, sensitive and quantitative analysis of proteins or peptide fractions in heterogenous mixtures of proteins. These analytical reagents, herein referred to as “dye reagents” or “stable isotope dye reagents” have the formula:

D-L-R

wherein D is a dye moiety, L is a linker and R is a reactive group that selectively reacts with a functional group of a protein wherein the dye moiety or linker contains at least one stable isotope. The dye moiety or linker or both can contain stable isotopes wherein “light” isotopes have been replaced with “heavy” isotopes. These “heavy” isotopes that find particular use in this invention are selected from the group consisting of ²H, ¹³C, ¹⁵N, ¹⁷O, ¹⁸O, ¹⁸F and ³⁴S.

The dye moiety (D) of the present invention confers a detectable signal, directly or indirectly, to the tagged proteins resulting in the ability to visually detect and monitor the tagged proteins.

The dye moiety includes, but is not limited to, the group consisting of xanthene, borapolyazaindacene, cyanine, coumarin, acridine, furan, indole, quinoline, benzofuran, quinazolinone, and benzazole. The xanthene moieties are selected from the group consisting of fluorescein, rhodamine, rosamine, rhodol and derivatives thereof.

The reactive group of the dye reagent is a group that will selectively reactive with a protein functional group. These groups include amine, thiol, ketone, and alcohol. In one aspect the reactive group is selected from the group consisting of carboxylic acid, succinimidyl ester of a carboxylic acid, hydrazide, amine, tetrafluorophenyl ester, isothiocyanate, sulfonyl chloride, a photoactivatable group or a maleimide.

In addition, the dye reagent can comprise a second reactive group, wherein the reactive group typically functions to covalently attach the optional affinity tag. The affinity tag employed to separate non-labeled proteins from a mixture of labeled proteins. These affinity tags include, but are not limited to, a hapten, glutathione, a metal chelating moiety, protein A, protein G and maltose. In a particular aspect the affinity reagent is biotin and its derivatives thereof.

The dye reagent comprises L (linker) that is a single covalent bond or a covalent linkage that is linear or branched, cyclic or heterocyclic, saturated or unsaturated, having 1-20 nonhydrogen atoms selected from the group consisting of C, N, P, O and S; and are composed of any combination of ether, thioether, amine, ester, carboxamide, sulfonamide, hydrazide bonds and aromatic or heteroaromatic bonds. In one aspect the linker contains a cleavable moiety.

These dye reagents are an improvement over currently used ICAT reagents and can be used in place of those current reagents for increased sensitivity of differentially labeled proteins using methods known in the art (U.S. Pat. No. 6,670,194 and US 2004/0106150). In addition, the use of a dye moiety allows for more flexible multiplexing wherein dye moieties that absorb and emit at distinguishable wavelengths can be employed for this purpose.

Thus, in one aspect, the methods of using the dye reagents for the identifying and determining of the relative amounts of one or more proteins in two or more samples, comprises the steps:
a) contacting each sample with a dye reagent that is substantially chemically identical but isotopically distinguishable, wherein the dye reagent has the formula:

D-L-R
wherein D is a dye moiety, L is a linker and R is a reactive group that selectively reacts with a functional group of a protein wherein either the dye moiety or the linker or both are labeled with one or more stable isotopes;
b) incubating each sample with the isotopically distinguishable dye reagent to provide discrete sets of dye reagent tagged proteins, dye reagent tagged proteins in different samples being thereby differently labeled with one or more stable isotopes;
c) combining the discrete sets of differentially labeled samples to provide a pooled labeled sample; and,
d) detecting, measuring and determining the pooled differentially labeled proteins whereby the relative amounts of proteins are identified and determined.

In another embodiment is provided a kit for the identification and determination of the relative amounts of one or more proteins in two or more samples. In one aspect the kit comprises a first dye reagent comprising at least one stable isotope wherein the reagent has the formula D-L-R: wherein D is a dye moiety, L is a linker and R is a reactive group that selectively reacts with a functional group of a protein; and a second dye reagent that is substantially chemically identical to the first dye reagent but isotpically distinguishable.

### BRIEF DESCRIPTION OF THE FIGURES

FIG. 1 shows the MALDI analysis results of the co-mixture of equal amounts of heavy and light labeled AT1 (lower panel), as well as, the individual heavy and light labeled reagents (upper panels).

### DETAILED DESCRIPTION OF THE INVENTION

### Introduction

The present invention includes novel reactive fluorescent compounds that incorporate stable isotopic (deuterium, 13-carbon, 15-nitrogen, 18-oxygen) substitutions. The invention includes the use of these compounds, in combination with non-isotopically substituted analogs, for the purification, identification and relative quantification of proteins, peptides, saccharides, metabolites, and other biologically important compounds by combining liquid chromatography (LC) and mass spectrometry (MS). Fluorescent labeling of target compounds in this manner provides orders-of-magnitude sensitivity enhancement over traditional stable isotope labels, and also affords the possibility of simultaneous multiplexed analysis due to the multiwavelength nature of different fluorophores.

### DEFINITIONS

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a present compound” includes a plurality of compounds and reference to “a fluorophore” includes a plurality of ions and the like.

Unless defined otherwise, all technical and scientific terms used herein have the same meaning as commonly understood by one of ordinary skill in the art to which this invention is related. The following terms are defined for purposes of the invention as described herein.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL's ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The compounds of the present invention contain unnatural proportions of atomic isotopes at one or more of the atoms that constitute such compounds. For example, the compounds are labeled with stable isotopes, such as for example deuterium (²H), nitrogen (¹⁵N), oxygen (¹⁸O), or carbon-13 (¹³C). All isotopic variations of the compounds of the present invention are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical” is the group derived from a carboxylic acid by removing the —OH moiety therefrom.

The term “alkyl,” by itself or as part of another substituent means, unless otherwise stated, a straight or branched chain, or cyclic hydrocarbon radical, or combination thereof, which may be fully saturated, mono- or polyunsaturated and can include divalent (“alkylene”) and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present invention contain between about one and about twenty-five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom selected from the group consisting of O, N, Si, P and S, and wherein the nitrogen, phosphorous and sulfur atoms are optionally oxidized, and the nitrogen heteroatom is optionally be quaternized. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic moiety that can be a single ring or multiple rings (preferably from 1 to 3 rings), which are fused together or linked covalently.

The term “heteroaryl” as used herein refers to an aryl group as defined above in which one or more carbon atoms have been replaced by a non-carbon atom, especially nitrogen, oxygen, or sulfur. For example, but not as a limitation, such groups include furyl, tetrahydrofuryl, pyrrolyl, pyrrolidinyl, thienyl, tetrahydrothienyl, oxazolyl, isoxazolyl, triazolyl, thiazolyl, isothiazolyl, pyrazolyl, pyrazolidinyl, oxadiazolyl, thiadiazolyl, imidazolyl, imidazolinyl, pyridyl, pyridaziyl, triazinyl, piperidinyl, morpholinyl, thiomorpholinyl, pyrazinyl, piperainyl, pyrimidinyl, naphthyridinyl, benzofuranyl, benzothienyl, indolyl, indolinyl, indolizinyl, indazolyl, quinolizinyl, qunolinyl, isoquinolinyl, cinnolinyl, phthalazinyl, quinazolinyl, quinoxalinyl, pteridinyl, quinuclidinyl, carbazolyl, acridinyl, phenazinyl, phenothizinyl, phenoxazinyl, purinyl, benzimidazolyl and benzthiazolyl and their aromatic ring-fused analogs. Many fluorophores are comprised of heteroaryl groups and include, without limitations, xanthenes, oxazines, benzazolium derivatives (including cyanines and carbocyanines), borapolyazaindacenes, benzofurans, indoles and quinazolones.

The above heterocyclic groups may further include one or more substituents at one or more carbon and/or non-carbon atoms of the heteroaryl group, e.g., alkyl; aryl; heterocycle; halogen; nitro; cyano; hydroxyl, alkoxyl or aryloxyl; thio or mercapto, alkyl- or arylthio; amino, alkyl-, aryl-, dialkyl-, diaryl-, or arylalkylamino; aminocarbonyl, alkylaminocarbonyl, arylaminocarbonyl, dialkylaminocarbonyl, diarylaminocarbonyl or arylalkylaminocarbonyl; carboxyl, or alkyl- or aryloxycarbonyl; aldehyde; aryl- or alkylcarbonyl; iminyl, or aryl- or alkyliminyl; sulfo; alkyl- or arylsulfonyl; hydroximinyl, or aryl- or alkoximinyl. In addition, two or more alkyl substituents may be combined to form fused heterocycle-alkyl ring systems. Substituents including heterocyclic groups (e.g., heteroaryloxy, and heteroaralkylthio) are defined by analogy to the above-described terms.

The term “heterocycloalkyl” as used herein refers to a heterocycle group that is joined to a parent structure by one or more alkyl groups as described above, e.g., 2-piperidylmethyl, and the like. The term “heterocycloalkyl” refers to a heteroaryl group that is joined to a parent structure by one or more alkyl groups as described above, e.g., 2-thienylmethyl, and the like.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R″′ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R′, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R″′ and R″″ groups when more than one of these groups is present. In the schemes that follow, the symbol X represents “R” as described above.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “amino” or “amine group” refers to the group —NR′R″ (or NRR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —NRR′R″ and its biologically compatible anionic counterions.

The term “aqueous solution” as used herein refers to a solution that is predominantly water and retains the solution characteristics of water. Where the aqueous solution contains solvents in addition to water, water is typically the predominant solvent.

The term “buffer” as used herein refers to a system that acts to minimize the change in acidity or basicity of the solution against addition or depletion of chemical substances.

The term “carbonyl” as used herein refers to the functional group —(C═O)—. However, it will be appreciated that this group may be replaced with other well-known groups that have similar electronic and/or steric character, such as thiocarbonyl (—(C═S)—); sulfinyl (—S(O)—); sulfonyl (—SO₂)—), phosphonyl (—PO₂—).

The term “carboxy” or “carboxyl” refers to the group —R′(COOR) where R′ is alkyl, substituted alkyl, aryl, substituted aryl, arylalkyl, substituted arylalkyl, heteroaryl, or substituted heteroaryl. R is hydrogen, a salt or —CH₂OC(O)CH₃.

The term “detectable response” as used herein refers to a change in or an occurrence of, a signal that is directly or indirectly detectable either by observation or by instrumentation. Typically, the detectable response is an optical response resulting in a change in the wavelength distribution patterns or intensity of absorbance or fluorescence or a change in light scatter, fluorescence lifetime, fluorescence polarization, or a combination of the above parameters. Alternatively, the detectable response is an occurrence of a signal wherein the fluorophore is inherently fluorescent and does not produce a change in signal upon binding to a metal ion.

The term “differentially-expressed” as used herein refers to the quantitative changes in expression level of protein(s), as well as qualitative changes such as covalent changes, e.g., post-translational modifications such as protein phosphorylation, protein glycosylation, protein acetylation and protein processing of the C- or N-terminal of a protein.

The term “directly detectable” as used herein refers to the presence of a detectable label or the signal generated from a detectable label that is immediately detectable by observation, instrumentation, or film without requiring chemical modifications or additional substances. For example, a fluorophore produces a directly detectable response.

The term “dye” as used herein refers to a compound that emits light to produce an observable detectable signal. “Dye” includes fluorescent and nonfluorescent compounds that include without limitations pigments, fluorophores, chemiluminescent compounds, luminescent compounds and chromophores. The term “fluorophore” as used herein refers to a compound that is inherently fluorescent or demonstrates a change in fluorescence upon binding to a biological compound or metal ion, i.e., fluorogenic. Numerous fluorophores are known to those skilled in the art and include, but are not limited to, coumarin, acridine, furan, indole, quinoline, cyanine, benzofuran, quinazolinone, benzazole, borapolyazaindacene and xanthenes, with the latter including fluoroscein, rhodamine, rhodol, rosamine and derivatives thereof as well as other fluorophores described in RICHARD P. HAUGLAND, MOLECULAR PROBES HANDBOOK OF FLUORESCENT PROBES AND RESEARCH CHEMICALS (9^{th} edition, CD-ROM, 2002).

The term “ICAT” as used herein refers to isotope coded affinity tag.

The term “kit” as used herein refers to a packaged set of related components, typically one or more compounds or compositions.

The term “Linker” or “L”, as used herein, refers to a single covalent bond or a series of stable covalent bonds incorporating 1-30 nonhydrogen atoms selected from the group consisting of C, N, O, S and P that covalently attach the phosphate-binding compounds to another moiety such as a chemically reactive group or a phosphorylated target molecule. Exemplary linking members include a moiety that includes —C(O)NH—, —C(O)O—, —NH—, —S—, —O—, and the like. A “cleavable linker” is a linker that has one or more cleavable groups that may be broken by the result of a reaction or condition. The term “cleavable group” refers to a moiety that allows for release of a portion, e.g., a reporter molecule, carrier molecule or solid support, of a conjugate from the remainder of the conjugate by cleaving a bond linking the released moiety to the remainder of the conjugate. Such cleavage is either chemical in nature, or enzymatically mediated. Exemplary enzymatically cleavable groups include natural amino acids or peptide sequences that end with a natural amino acid.

In addition to enzymatically cleavable groups, it is within the scope of the present invention to include one or more sites that are cleaved by the action of an agent other than an enzyme. Exemplary non-enzymatic cleavage agents include, but are not limited to, acids, bases, light (e.g., nitrobenzyl derivatives, phenacyl groups, benzoin esters), and heat. Many cleaveable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989). Moreover a broad range of cleavable, bifunctional (both homo- and hetero-bifunctional) spacer arms are commercially available.

An exemplary cleavable group, an ester, is cleavable group that may be cleaved by a reagent, e.g. sodium hydroxide, resulting in a carboxylate-containing fragment and a hydroxyl-containing product.

The terms “protein” and “polypeptide” are used herein in a generic sense to include polymers of amino acid residues of any length. The term “peptide” is used herein to refer to polypeptides having less than 250 amino acid residues, typically less than 100 amino acid residues, more typically less than 15 amino acid residues. The terms apply to amino acid polymers in which one or more amino acid residues are an artificial chemical analogue of a corresponding naturally occurring amino acid, as well as to naturally occurring amino acid polymers. The peptide or protein may be further conjugated to or complexed with other moieties such as dyes, haptens, radioactive isotopes, natural and synthetic polymers (including microspheres), glass, metals and metallic particles, proteins and nucleic acids.

The term “reactive group” as used herein refers to a group that is capable of reacting with another chemical group to form a covalent bond, i.e. is covalently reactive under suitable reaction conditions, and generally represents a point of attachment for another substance. The reactive group is a moiety, such as a photoactivatable group, carboxylic acid or succinimidyl ester, on the compounds of the present invention that is capable of chemically reacting with a functional group on a different compound to form a covalent linkage resulting in a labeled protein, peptide or affinity reagent. Reactive groups generally include nucleophiles, electrophiles and photoactivatable groups.

Exemplary reactive groups include, but not limited to, epoxides, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare affinity reagents, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

The term “salt thereof,” as used herein includes salts of the agents of the invention and their conjugates, which are preferably prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of base addition salts include sodium, potassium, calcium, ammonium, organic amino, or magnesium, or a similar salt. When compounds of the present invention contain relatively basic functionalities, acid addition salts can be obtained by contacting the neutral form of such compounds with a sufficient amount of the desired acid, either neat or in a suitable inert solvent. Examples of addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “sample” as used herein refers to any material that may contain an analyte of interest and is intended to included the term in its broadest sense. Suitable samples include, but are not limited to, recombinant proteins over expressed in cells that are in the form of inclusion bodies or secreted from cells, normal and diseased cells, cell homogenates (cell lysates); cell fractions; tissue homogenates (tissue lysates); immunoprecipitates, biological fluids, such as blood, urine and cerebrospinal fluid; tears; feces; saliva; and lavage fluids, such as lung or peritoneal lavages. Typically, the sample is a cell extract or a biological fluid that comprises endogenous host cell proteins or expressed recombinant proteins.

The term “stable isotope” as used herein refers to a non-radioactive isotopic form of an element. Exemplary stable isotopes include ²H, ¹³C, ¹⁵N, ¹⁷O, ¹⁸O, ¹⁸F and ³⁴S.

### The Compounds

In general, for ease of understanding the present invention, the fluorescent isotope compounds and corresponding substituents will first be described in detail, followed by the many and varied methods in which the compounds find uses, which is followed by exemplified methods of use and synthesis of certain novel compounds that are particularly advantageous for use with the methods of the present invention.

This invention provides analytical reagents and mass spectrometry-based methods using these reagents for the rapid, sensitive, and quantitative analysis of proteins in mixtures of proteins. The analytical method can be used for qualitative and particularly for quantitative analysis of global protein expression profiles in cells and tissues, i.e. the quantitative analysis of proteomes. The method can also be employed to screen for and identify proteins whose expression level in cells, tissue or biological fluids is affected by a stimulus (e.g., administration of a drug or contact with a potentially toxic material), by a change in environment (e.g., nutrient level, temperature, passage of time) or by a change in condition or cell state (e.g., disease state, malignancy, site-directed mutation, gene knockouts) of the cell, tissue or organism from which the sample originated. The proteins identified in such a screen can function as markers for the changed state. For example, comparisons of protein expression profiles of normal and malignant cells can result in the identification of proteins whose presence or absence is characteristic and diagnostic of the malignancy.

These methods of the present invention employ isotopically labeled dye reagent tagged proteins for the analysis of two or more biological samples. The improvement over known isotopically labeled reagents is the addition of a dye moiety, which allows for sensitive detection of the differentially labeled proteins. The present dye reagent has the formula: D-L-R wherein D is a dye moiety, L is a linker and R is a reactive group that selectively reacts with a functional group of a protein. The linker or dye moiety or both can be isotopically labeled to generate pairs or discrete sets of reagents that are substantially chemically identical, but which are distinguishable by mass. For example, any one or more of the hydrogen, carbon, nitrogen, oxygen, sulfur, or fluorine atoms in the linker or dye moiety may be replaced with their isotopically stable isotopes including ²H, ¹³C, ¹⁵N, ¹⁷O, ¹⁸O, ¹⁸F and ³⁴S. Alternatively, discrete sets of reagents are chemically distinct wherein the dye moiety is optically distinguishable, multiplying the number of sets of reagents for multiplexing purposes.

In certain instances the dye reagent further comprises an affinity tag that facilitates isolation of the differently labeled samples.

### Dye Moieties

The dye moieties of the present invention confer a detectable signal, directly or indirectly, to the tagged proteins increasing the sensitivity of the isotopically labeled reagents. This results in the ability to visually detect and monitor the tagged proteins.

Thus, in an exemplary embodiment, a dye moiety is covalently bound to a reactive group via a linker. In another embodiment, the dye moiety contains a second reactive group that is utilized to covalently attach an affinity moiety to the present dye reagent. The reactive group may contain both a reactive functional moiety and a linker, or only the reactive functional moiety.

The dye moiety can be any dye moiety known to one skilled in the art and when covalently linked to a reactive group forms a dye reagent of the invention that is useful for analyzing proteins that are part of a complex heterogeneous mixture. Dye moieties include, without limitation, a chromophore, a fluorophore, a fluorescent protein, a phosphorescent dye, and a tandem dye (energy transfer pair). Preferred dye moieties include chromophores or fluorophores.

A dye of the present invention is any chemical moiety that exhibits an absorption maximum beyond 280 nm. Dyes of the present invention include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a carbocyanine (including any corresponding compounds in U.S. Ser. Nos. 09/557,275; 09/968,401 and 09/969,853 and U.S. Pat. Nos. 6,403,807; 6,348,599; 5,486,616; 5,268,486; 5,569,587; 5,569,766; 5,627,027 and 6,048,982), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and U.S. Ser. No. 09/922,333), an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

Where the dye is a xanthene, the dye is optionally a fluorescein, a rhodol (including any corresponding compounds disclosed in U.S. Pat. Nos. 5,227,487 and 5,442,045), a rosamine or a rhodamine (including any corresponding compounds in U.S. Pat. Nos. 5,798,276; 5,846,737; 5,847,162; 6,017,712; 6,025,505; 6,080,852; 6,716,979; 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171).

Preferred dyes of the invention include the xanthene moieties such as rhodol, fluorescein, rhodamine, and their derivatives.

Typically the dye contains one or more aromatic or heteroaromatic rings, that are optionally substituted one or more times by a variety of substituents, including without limitation, halogen, nitro, sulfo, cyano, alkyl, perfluoroalkyl, alkoxy, alkenyl, alkynyl, cycloalkyl, arylalkyl, acyl, aryl or heteroaryl ring system, benzo, or other substituents typically present on chromophores or fluorophores known in the art.

In an exemplary embodiment, the dyes are independently substituted by substituents selected from the group consisting of hydrogen, halogen, amino, substituted amino, alkyl, substituted alkyl, aryl, substituted aryl, heteroaryl, substituted heteroaryl, alkoxy, sulfo, reactive group and carrier molecule. In another embodiment, the xanthene dyes of this invention comprise both compounds substituted and unsubstituted on the carbon atom of the central ring of the xanthene by substituents typically found in the xanthene-based dyes such as phenyl and substituted-phenyl moieties. Most preferred dyes are rhodamine, fluorescein, rhodal, rosamine and derivatives thereof. The choice of the dye attached to the reactive group will determine the dye reagent absorption and fluorescence emission properties.

At least some of the atoms in the dye moiety should be readily replaceable with stable heavy-atom isotopes. The dye moiety preferably contains groups or moieties that facilitate ionization of the dye reagent tagged protein and/or peptides. To promote ionization, the dye moiety may contain acidic or basic groups, e.g., COOH, SO₃H, primary, secondary or tertiary amino groups, nitrogen-heterocycles, ethers, or combinations of these groups. The dye moiety may also contain groups having a permanent charge, e.g., phosphonium groups, quaternary ammonium groups, sulfonium groups, chelated metal ions, tetralky or tetraryl borate or stable carbanions.

In an exemplary embodiment, the dye has an absorption maximum beyond 480 nm. In a particularly useful embodiment, the dye absorbs at or near 488 nm to 514 nm (particularly suitable for excitation by the output of the argon-ion laser excitation source) or near 546 nm (particularly suitable for excitation by a mercury arc lamp). As is the case for many dyes, they can also function as both chromophores and fluorophores. Thus, the described fluorescent dyes are also the preferred chromophores of the present invention.

### Linkers

The linker group (L) should be soluble in the sample liquid to be analyzed and it should be stable with respect to chemical reaction, e.g., substantially chemically inert, with components of the sample. The linker when bound to D (dye moiety) should not interfere with the specific interaction of R (reactive group) or the optional affinity tag. The linker should bind minimally or preferably not at all to other components in the system or to reaction vessel surfaces. Any non-specific interactions of the linker should be disrupted after multiple washes, which leave the tagged protein complex intact. Linkers preferably do not undergo peptide-like fragmentation during MS analysis. At least some of the atoms in the linker groups should be readily replaceable with stable heavy-atom isotopes, as described above for the dye moiety.

The dye moiety and reactive group are directly attached (where Linker is a single bond) or attached through a series of stable bonds to form a present dye reagent having the formula D-L-R. When the linker is a series of stable covalent bonds the linker typically incorporates 1-30 nonhydrogen atoms selected from the group consisting of C, N, O, S and P. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. Typically the linker incorporates less than 15 nonhydrogen atoms and are composed of any combination of ether, thioether, thiourea, amine, ester, carboxamide, sulfonamide, hydrazide bonds and aromatic or heteroaromatic bonds. Typically the linker is a combination of single carbon-carbon bonds and carboxamide, sulfonamide or thioether bonds. The bonds of the linker typically result in the following moieties that can be found in the linker: ether, thioether, carboxamide, thiourea, sulfonamide, urea, urethane, hydrazine, alkyl, aryl, heteroaryl, alkoky, cycloalkyl and amine moieties. Examples of a linker include substituted or unsubstituted polymethylene, arylene, alkylarylene, arylenealkyl, or arylthio.

The linker preferably contains groups or moieties that facilitate ionization of the dye tagged reagents, protein or peptides. To promote ionization, the linker may contain acidic or basic groups, e.g., COOH, SO₃H, primary, secondary or tertiary amino groups, nitrogen-heterocycles, ethers, or combinations of these groups. The linker may also contain groups having a permanent charge, e.g., phosphonium groups, quaternary ammonium groups, sulfonium groups, chelated metal ions, tetralky or tetraryl borate or stable carbanions.

The covalent bond of the linker to D or R should typically not be unintentionally cleaved by chemical or enzymatic reactions during the assay. However, in some cases it may be desirable to cleave the linker from the dye moiety or the affinity tag, if present, or from the reactive group, for example to facilitate release from an affinity column or for sequencing purposes.

By “cleavable moiety” is meant a group that can be chemically, photochemically, thermal or enzymatically cleaved. Photocleavable groups in the linker may include the 1-(2-nitrophenyl)-ethyl group. Thermally labile linkers may, for example, be a double-stranded duplex formed from two complementary strands of nucleic acid, a strand of a nucleic acid with a complementary strand of a peptide nucleic acid, or two complementary peptide nucleic acid strands which will dissociate upon heating. Cleavable linkers also include those having disulfide bonds, acid or base labile groups, including among others, diarylmethyl or trimethylarylmethyl groups, silyl ethers, carbamates, oxyesters, thiesters, thionoesters, and α-fluorinated amides and esters. Enzymatically cleavable linkers can contain, for example, protease-sensitive amides or esters, β-lactamase-sensitive β-lactam analogs and linkers that are nuclease-cleavable, or glycosidase-cleavable.

In one embodiment of the invention, the cleavable moiety is a moiety that forms a stable bond but can be efficiently cleaved under mild, preferably physiological, conditions. In a preferred embodiment, the cleavage site utilizes a photocleavable moiety. That is, upon exposure to suitable wavelengths of light absorbed by the photo-cleavable groups, cleavage of the linker occurs, thereby removing the dye from the protein or other molecule to facilitate further analysis. In one aspect the photocleavable moieties are the O-nitrobenzylic compounds, which can be synthetically incorporated into the dye reagent via an ether, thioether, ester (including phosphate esters), amine or similar linkage to a heteroatom (particularly oxygen, nitrogen or sulfur). Also of use are benzoin-based photocleavable moieties. Nitrophenylcarbamate esters are particularly preferred. A wide variety of suitable photocleavable moieties is outlined in The Molecular Probes Handbook, Tenth Edition 2005.

By engineering in a cleavable moiety on the optical labeling molecule, the maximum detection sensitivity of the labeling molecule is increased by allowing a high multiplicity of dye labeling that will increase the maximum detection sensitivity, followed by removal of the labeling molecule prior to further analysis. For example, the dye reagent can be removed after protein separation via cleavage of the cleavable moiety prior to mass spectroscopy (MS) analysis. Identification of interesting protein spots on 2D gels for further study is typically accomplished by fluorescent scanning during analysis of the gels, but identification of the proteins contained in those spots is generally accomplished by mass spectrometry. The most generally effective method of identifying proteins and post-translational modifications digests proteins with trypsin or other lysine-specific enzymes, before analysis by mass spectrometry. As is well known in the art, trypsin is an enzyme that specifically cleaves at the basic amino acid groups, arginine and lysine. High multiplicity attachment of dye reagent on amino groups will “cover” some of the most accessible lysine amino groups and if the dyes are not removed they will inhibit trypsin digestion at these sites. In some embodiments, this may be preferred. Thus, the removal of the dye after protein separation by chemical, photochemical or enzymatic cleavage is preferable in some embodiments.

Typically the stable isotopes are between the cleavable moiety and the reactive group. With this embodiment, when the cleavable moiety is cleaved, the stable isotope moieity is left on the protein and the relative amount of the protein expressed by the biological system under different stimulus conditions can be quantitated using isotope ratios in a mass spectrometer.

### Reactive Groups

The dye reagents of the present invention comprise at least one reactive group that selectively reacts with functional groups commonly found on proteins. Thus, the present dye reagents are chemically reactive. Typically the present compounds comprise thiol- or amine-reactive groups. However, reactive groups that selectively react with other functional groups such as alcohols, acrylamides, sugars, and phosphates that may be present or induced in proteins are also contemplated. Any selectively reactive protein reactive group should react with a functional group of interest that is present in at least a portion of the proteins in a sample. Reaction of a reactive group with functional groups on the protein should occur under conditions that do not lead to substantial degradation of the compounds in the sample to be analyzed.

Alternatively, the present dye reagent comprises a second reactive group that facilitates covalent attachment of an affinity tag to the dye moiety. The selection of the second reactive group will, in part, be determined by the affinity tag to be conjugated and the corresponding functional groups present on the affinity tag. Thus, a wide variety of reactive groups are contemplated for the present inventions, which can be generally grouped into three categories: electrophile, nucleophile and photoactivatable group.

In an exemplary embodiment, the compounds of the invention comprise a reactive group which is a member selected from an acrylamide, an activated ester of a carboxylic acid, a carboxylic ester, an acyl azide, an acyl nitrile, an aldehyde, an alkyl halide, an anhydride, an aniline, an amine, an aryl halide, an azide, an aziridine, a boronate, a diazoalkane, an epoxide, a haloacetamide, a halotriazine, a hydrazine, a hydroxylamine, an imido ester, an isocyanate, an isothiocyanate, a maleimide, a phosphoramidite, a photoactivatable group, a reactive platinum complex, a silyl halide, a sulfonyl halide, and a thiol. In a particular embodiment the reactive group is selected from the group consisting of carboxylic acid, succinimidyl ester of a carboxylic acid, hydrazide, hydroxylamine, amine and a haloacetamide.

In an exemplary embodiment, the compound comprises at least one reactive group that selectively reacts with an amine group. This amine-reactive group is selected from the group consisting of succinimidyl ester, sulfonyl halide, perfluorophenyl ester and iosothiocyanates.

In another exemplary embodiment, the compounds comprise at least one reactive group that selectively reacts with a thiol group. This thiol-reactive group is selected from the group consisting of acrylamide, alkyl halide, haloacetamide, maleimide, and epoxide. Thus, in one aspect, the present compounds form a covalent bond with a thiol-containing molecule in a sample such as proteins or peptides.

In another exemplary embodiment, the compounds comprise at least one reactive group that selectively reacts with the reducing end of a saccharide, or an aldehyde, group. This carbonyl-reactive group is selected from the group consisting of hydrazine, hydrazide, thiosemicarbazide, and hydroxylamine. Thus, in one aspect, the present compounds form a covalent bond with an aldehyde-containing molecule in a sample such as proteins or peptides or saccharides or oligosaccharides.

These reactive groups are synthesized during the synthesis of the dye moiety to provide chemically reactive fluorescent isotope labeled compounds. In an exemplary embodiment, the reactive group of the compounds of the invention and the functional group of the protein comprise electrophiles and nucleophiles that can generate a covalent linkage between them. Alternatively, the reactive group comprises a photoactivatable group, which becomes chemically reactive only after illumination with light of an appropriate wavelength. Typically, the conjugation reaction between the reactive group and the sample results in one or more atoms of the reactive group being incorporated into a new linkage attaching the dye moiety of the invention to the protein or peptide. Selected examples of functional groups and linkages are shown in Table 1, where the reaction of an electrophilic group and a nucleophilic group yields a covalent linkage.

TABLE 1
Examples of some routes to useful covalent linkages

| Electrophilic Group | Nucleophilic Group | Resulting Covalent Linkage |
|---|---|---|
| activated esters* | amines/anilines | carboxamides |
| acrylamides | thiols | thioethers |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

*Activated esters, as understood in the art, generally have the formula —COΩ, where Ω is a good leaving group (e.g., succinimidyloxy (—OC₄H₄O₂) sulfosuccinimidyloxy (—OC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl).
**Acyl azides can also rearrange to isocyanates

Choice of the reactive group used to attach the compound of the invention to the substance to be tagged typically depends on the reactive or functional group on the substance to be conjugated and the type or length of covalent linkage desired. The types of functional groups typically present on protein substances or which react with groups on proteins include, but are not limited to, amines, amides, thiols, alcohols, phenols, aldehydes, ketones, phosphates, imidazoles, hydrazines, hydroxylamines, disubstituted amines, halides, epoxides, silyl halides, carboxylate esters, sulfonate esters, carboxylic acids, olefinic bonds, or a combination of these groups. A single type of reactive site may be available on the substance (typical for polysaccharides), or a variety of sites may occur (e.g., amines, thiols, alcohols, phenols), as is typical for proteins.

Typically, the reactive group will react with an amine, a thiol, an alcohol, an aldehyde, or a ketone. Preferably, reactive groups react with an amine, a thiol, or an aldehyde functional group. In one embodiment, the reactive group is an acrylamide, an activated ester of a carboxylic acid, an acyl azide, an acyl nitrile, an aldehyde, an alkyl halide, a silyl halide, an anhydride, an aniline, an aryl halide, an azide, an aziridine, a boronate, a diazoalkane, a haloacetamide, a halotriazine, a hydrazine (including hydrazides), an imido ester, an isocyanate, an isothiocyanate, a maleimide, a phosphoramidite, a sulfonyl halide, or a thiol group.

Where the reactive group is an activated ester of a carboxylic acid, such as a succinimidyl ester of a carboxylic acid, a sulfonyl halide, a tetrafluorophenyl ester or an isothiocyanates, the resulting compound is particularly useful for labeling proteins or haptens. Where the reactive group is a maleimide or haloacetamide the resulting compound is particularly useful for conjugation to thiol-containing substances. Where the reactive group is a hydrazide, the resulting compound is particularly useful for conjugation to periodate-oxidized carbohydrates and glycoproteins.

In a particular aspect, the reactive group is a photoactivatable group such that the group is only converted to a reactive species after illumination with an appropriate wavelength. An appropriate wavelength is generally a UV wavelength that is less than 400 nm. This method provides for specific attachment to only the target molecules, either in solution or immobilized on a solid or semi-solid matrix. Photoactivatable reactive groups include, without limitation, benzophenones, aryl azides and diazirines.

Preferably, the reactive group is an acrylamide, an activated ester of a carboxylic acid, a carboxylic ester, an acyl azide, an acyl nitrile, an aldehyde, an alkyl halide, an anhydride, an aniline, an amine, an aryl halide, an azide, an aziridine, a boronate, a diazoalkane, an epoxide, a haloacetamide, a halotriazine, a hydrazine, an imido ester, an isocyanate, an isothiocyanate, a maleimide, a phosphoramidite, a reactive platinum complex, a silyl halide, a sulfonyl halide, a thiol and a photoactivatable group. In a particular embodiment the reactive group is a succinimidyl ester of a carboxylic acid, a sulfonyl halide, a tetrafluorophenyl ester, an iosothiocyanates or a maleimide.

### Affinity Tags

For purposes of isolation the present dye reagent optionally comprises an affinity tag. The affinity tag may be covalently attached to the dye moiety or linker though a conjugation reaction facilitated by a reactive group. Alternatively, the affinity tags, typically non-protein moieties, are covalently attached to the dye reagent during synthesis of the reagent.

Suitable affinity tags bind selectively either covalently or non-covalently and with high affinity to a capture reagent. The capture reagent affinity tag interaction or bond should remain intact after extensive and multiple washings with a variety of solutions to remove non-specifically bound components. The affinity tag binds minimally or preferably not at all to components in the assay system, except for the exogenously added capture reagent, and does not significantly bind to surfaces of reaction vessels. Any non-specific interaction of the affinity tag with other components or surfaces should be disrupted by multiple washes that leave the capture reagent affinity tag complex intact. Further, it must be possible to disrupt the interaction of the affinity tag and capture reagent to release peptides or protein, for example, by addition of a displacing ligand or by changing the temperature or solvent conditions. Preferably, neither the capture reagent nor the affinity tag react chemically with other components in the assay system and both groups should be chemically stable over the time period of an assay or experiment. The affinity tag preferably does not undergo peptide-like fragmentation during MS analysis. The affinity tag is preferably soluble in the sample liquid to be analyzed and the capture reagent should remain soluble in the sample liquid even though attached to an insoluble resin such as Agarose. In this instance soluble means that the capture reagent is sufficiently hydrated or otherwise solvated such that it functions properly for binding to the affinity tag. Capture reagents or capture reagent-containing conjugates should not be present in the sample to be analyzed, except when added exogenously to the sample.

A variety of affinity tags are useful in the present invention. Exemplary affinity tags include haptens, antigens, oligohistidine sequences, metal chelating moieties, glutathione, maltose, streptavidin, protein A, protein G and other ligands.

Ligands that bind transitional metals include metal chelating moieties such as BAPTA, IDA, APTRA, NTA, DTPA, TTHA, and crown ether and oligomeric histidine. The capture reagent may either be chelating moiety or a different ligand such as a histidine tag or a metal ion that is covalently attached to a solid support such as agarose.

Biotin and biotin-based affinity tags are preferred. A preferred form of biotin is the desthiobiotin analog, which can be easily adsorbed and released from avidin-based affinity matrices. A preferred form of avidin for some applications is CaptAvidin biotin-binding protein (Molecular Probes), which permits facile release of biotinylated compounds. Of particular interest are structurally modified biotins, such as d-iminobiotin, which will elute from avidin or strepavidin columns under solvent conditions compatible with ESI-MS analysis, such as dilute acids containing 10-20% organic solvent. It is expected that d-iminobiotin tagged compounds will elute in solvents below pH 4.

Furthermore, haptens also include, among other derivatives, hormones, naturally occurring and synthetic drugs, pollutants, allergens, affector molecules, growth factors, chemokines, cytokines, lymphokines, amino acids, peptides, chemical intermediates, nucleotides and the like.

Other affinity tags include, maltose, which binds to maltose binding protein (as well as any other sugar/sugar binding protein pair or more generally to any ligand/ligand binding protein pairs that has properties discussed above); dinitrophenyl group, for any antibody where the hapten binds to an anti-hapten antibody that recognizes the hapten, for example the dinitrophenyl group will bind to an anti-dinitrophenyl-IgG; digoxigenin wherein commercially available antibodies that selectively bind digoxigenin exist; glutathione which binds to glutathione-S-transferase; protein A or an anti-Fc region antibody fragment which binds to the Fc portion of an antibody.

In general, any affinity tag-capture reagent pair commonly used for affinity enrichment which meets the suitability criteria discussed above, may be employed in the methods and dye reagents of the present invention. Exemplary binding pairs are set forth in Table 2.

TABLE 2
Representative Generic Affinity Tag-Capture Reagent Pairs

| antigen | antibody |
|---|---|
| biotin | avidin (or streptavidin or anti-biotin) |
| IgG | protein A or protein G |
| drug | drug receptor |
| maltose | Maltose binding protein |
| folate | folate binding protein |
| toxin | toxin receptor |
| carbohydrate | lectin or carbohydrate receptor |
| peptide | peptide receptor |
| protein | protein receptor |
| enzyme substrate | enzyme |
| DNA (RNA) | cDNA (cRNA) |
| hormone | hormone receptor |
| metal ion | chelator |

### Preparation of Affinity Tag Conjugates

Conjugates of affinity tags, e.g., protein, peptides, and other organic molecules are prepared by organic synthesis methods using the reactive dye reagents of the invention, are generally prepared by means well recognized in the art (Haugland, MOLECULAR PROBES HANDBOOK, supra, Sets 1-7, (1992)). Preferably, conjugation to form a covalent bond consists of simply mixing the reactive dyes of the present invention in a suitable solvent in which both the reactive dye and the affinity tag are soluble. The reaction preferably proceeds spontaneously without added reagents at room temperature or below. For those reactive dyes that are photoactivated, conjugation is facilitated by illumination of the reaction mixture to activate the reactive dye. Chemical modification of water-insoluble substances, so that a desired dye-conjugate may be prepared, is preferably performed in an aprotic solvent such as dimethylformamide, dimethylsulfoxide, acetone, ethyl acetate, toluene, or chloroform. Similar modification of water-soluble materials is readily accomplished through the use of the instant reactive dyes to make them more readily soluble in organic solvents. Many of the dyes of the present invention are readily dissolved in aqueous solution by adjusting the pH of the solution to about 6 or higher. Preparation of peptide or protein conjugates typically comprises first dissolving the protein to be conjugated in aqueous buffer at about. 1-10 mg/mL at room temperature or below. Bicarbonate buffers (pH about 8.3) are especially suitable for reaction with succinimidyl esters, phosphate buffers (pH about 7.2-8) for reaction with thiol-reactive functional groups and carbonate or borate buffers (pH about 9) for reaction with isothiocyanates and dichlorotriazines. The appropriate reactive dye is then dissolved in a nonhydroxylic solvent (usually DMSO or DMF) in an amount sufficient to give a suitable degree of conjugation when added to a solution of the protein to be conjugated. The appropriate amount of dye for any protein or other component is conveniently predetermined by experimentation in which variable amounts of the dye are added to the protein, the conjugate is chromatographically purified to separate unconjugated dye and the dye-affinity tag conjugate is tested in its desired application.

Following addition of the reactive dye to the component solution, the mixture is incubated for a suitable period (typically about 1 hour at room temperature to several hours on ice), the excess dye is removed by gel filtration, dialysis, HPLC, adsorption on an ion exchange or hydrophobic polymer or other suitable means. The dye-conjugate is used in solution or lyophilized. In this way, suitable conjugates can be prepared from antibodies, antibody fragments, avidins, lectins, enzymes, proteins A and G, and other affinity tags. The approximate degree of dye substitution is determined from the long wavelength absorption of the dye-affinity tag conjugate by using the extinction coefficient of the unreacted dye at its long wavelength absorption peak, the unmodified protein's absorption peak in the ultraviolet and by correcting the UV absorption of the conjugate for absorption by the dye in the UV.

Conjugates of polymers, including biopolymers and other higher molecular weight polymers are typically prepared by means well recognized in the art (for example, Brinkley et al., Bioconjugate Chem., 3: 2 (1992)). In these embodiments, a single type of reactive site may be available, as is typical for polysaccharides) or multiple types of reactive sites (e.g. amines, thiols, alcohols, phenols) may be available, as is typical for proteins. Selectivity of labeling is best obtained by selection of an appropriate reactive dye. For example, modification of thiols with a thiol-selective reagent such as a haloacetamide or maleimide, or modification of amines with an amine-reactive reagent such as an activated ester, acyl azide, isothiocyanate or 3,5-dichloro-2,4,6-triazine. Partial selectivity can also be obtained by careful control of the reaction conditions.

When modifying polymers with the dyes, an excess of dye is typically used, relative to the expected degree of dye substitution. Any residual, unreacted dye or a dye hydrolysis product is typically removed by dialysis, chromatography or precipitation. Presence of residual, unconjugated dye can be detected by thin layer chromatography using a solvent that elutes the dye away from its conjugate. In all cases it is usually preferred that the reagents be kept as concentrated as practical so as to obtain adequate rates of conjugation.

### Synthesis Scheme

For xanthene-based compounds of the present invention, stable isotope-substituted versions of conventional building blocks are required. Conventional building blocks include resorcinols, 3-aminophenols, benzaldehydes, benzoic acids and benzoyl halides, phthalic acids and anhydrides, and sulfobenzoic acids.

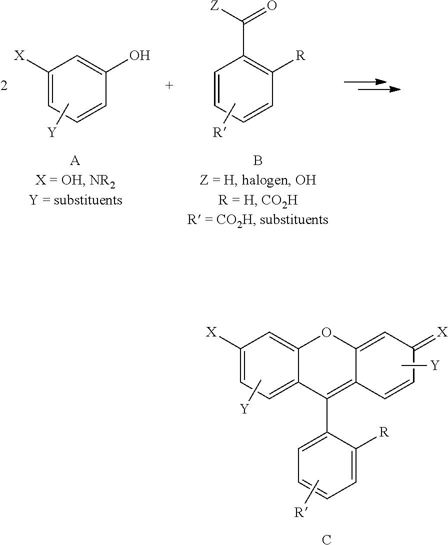

Acid mediated condensation of two equivalents of the phenol (A) an anti-Fc region antibody fragment) with one equivalent of the carbonyl benzene component (B) results in a tricyclic xanthene structure C. When X═NR₂ and R═CO₂H, the resulting xanthene dye C is a rhodamine. When X═NR₂ and R═H, the resulting xanthene dye C is a rosamine. When X═OH and R═CO₂H, the resulting xanthene dye C is a fluorescein. When Z═H in B, a dehydrogenative oxidation step is needed (for example mediated by p-chloranil) to produce C. Substituents Y, R, and R′ are chosen for the properties they impart to the final product C, including subsequent reactivity for conversion into reactive groups and/or incorporation of linker moieties.

Alternatively, C can be further derivatized by chemical reactions not involving substituents Y, R, or R′.

One way in which compounds of the present invention (C) are synthesized is by incorporating stable isotopes into either A and/or B. Another way in which compounds of the present invention are synthesized is by reacting a stable isotope-containing linker moiety with C; the resulting compound must be convertible into a reactive form.

### Methods of Use

The present invention also provides methods of using the compounds described herein to detect differently labeled analytes in a sample. Those of skill in the art will appreciate that this focus is for clarity of illustration and does not limit the scope of the methods in which the compounds of the invention find use.

The analytical methods of the invention can be used for qualitative and particularly for quantitative analysis of global protein expression profiles in cells and tissues, i.e. the quantitative analysis of proteomes. The method can also be employed to screen for and identify proteins whose expression level in cells, tissue or biological fluids is affected by a stimulus (e.g., administration of a drug or contact with a potentially toxic material), by a change in environment (e.g., nutrient level, temperature, passage of time) or by a change in condition or cell state (e.g., disease state, malignancy, site-directed mutation, gene knockouts) of the cell, tissue or organism from which the sample originated. The proteins identified in such a screen can function as markers for the changed state. For example, comparisons of protein expression profiles of normal and malignant cells can result in the identification of proteins whose presence or absence is characteristic and diagnostic of the malignancy.

The present dye reagents are an improvement over currently used ICAT reagents and can be used in place of those current reagents for increased sensitivity of differentially labeled proteins using methods known in the art (U.S. Pat. No. 6,670,194 and US 2004/0106150). In addition, the use of a dye moiety allows for more flexible multiplexing wherein dye moieties that absorb and emit at distinguishable wavelengths can be employed for this purpose.

Isolated labeled peptides according to the invention can be used to facilitate quantitative determination by mass spectrometry of the relative amounts of proteins in different samples. Also, the use of differentially isotopically-labeled reagents as internal standards facilitates quantitative determination of the absolute amounts of one or more proteins present in the sample. Samples that can be analyzed by method of the invention include, but are not limited to, cell homogenates; cell fractions; biological fluids, including, but not limited to urine, blood, and cerebrospinal fluid; tissue homogenates; tears; feces; saliva; ravage fluids such as lung or peritoneal ravages; and generally, any mixture of biomolecules, e.g., such as mixtures including proteins and one or more of lipids, carbohydrates, and nucleic acids such as obtained partial or complete fractionation of cell or tissue homogenates.

In a preferred embodiment, the type and number of proteins to be labeled will be determined by the method or desired result. In some instances, most or all of the proteins of a cell or virus are labeled; in other instances, some subset, for example subcellular fractionation, is first carried out, or macromolecular protein complexes are first isolated, as is known in the art, before dye labeling, protein separation and analysis.

In one embodiment a proteome is analyzed. By a proteome is intended at least about 20% of total protein coming from a biological sample source, usually at least about 40%, more usually at least about 75%, and generally 90% or more, up to and including all of the protein obtainable from the source. Thus the proteome may be present in an intact cell, a lysate, a microsomal fraction, an organelle, a partially extracted lysate, biological fluid, and the like. The proteome will be a mixture of proteins, generally having at least about 20 different proteins, usually at least about 50 different proteins and in most cases, about 100 different proteins or more.

Generally, the sample will have at least about 0.05 mg of protein or peptide, usually at least about 1 mg of protein or 10 mg of protein or more, typically at a concentration in the range of about 0.1-10 mg/ml. The sample may be adjusted to the appropriate buffer concentration and pH, if desired.

Thus, the present invention provides a method for identifying and determining the relative amounts of one or more proteins in two or more samples, which comprises the steps:
a) contacting each sample with a dye reagent that is substantially chemically identical but isotopically distinguishable, wherein said dye reagent has the formula:

D-L-R
wherein D is a dye moiety, L is a linker and R is a reactive group that selectively reacts with a functional group of a protein wherein either said dye moiety or said linker or both are labeled with one or more stable isotopes;
b) incubating each sample with the isotopically distinguishable dye reagent to provide discrete sets of dye reagent labeled proteins, dye reagent labeled proteins in different samples being thereby differently labeled with one or more stable isotopes;
c) combining the discrete sets of differentially labeled samples to provide a pooled labeled sample;
d) detecting, measuring and determining the pooled differentially labeled proteins whereby the relative amounts of proteins are identified and determined.

The present method uses matched pair dye reagents, wherein a first dye reagent is contacted with a sample and a second sample is contacted with a second dye reagent. The difference between the first and second dye reagents are the isotopic labels. An example of a matched pair of first and second dye reagents is exemplified as Compounds 39 and 42. Typically the sample comprises a target protein or target analyte wherein one sample is a reference and the other contains the target analyte.

Target proteins of the invention include all cellular proteins. In one embodiment, target proteins include regulatory proteins such as receptors and transcription factors as well as structural proteins.

In another embodiment target proteins include enzymes. As will be appreciated by those in the art, any number of different enzymes can be labeled. The enzymes (or other proteins) may be from any organisms, including prokaryotes and eukaryotes, with enzymes from bacteria, fungi, extremeophiles, viruses, animals (particularly mammals and particularly human) and birds all possible. Suitable classes of enzymes include, but are not limited to, hydrolases such as proteases, carbohydrases, lipases; isomerases such as racemases, epimerases, tautomerases, or mutases; transferases, kinases and phophatases. Preferred enzymes include those that carry out group transfers, such as acyl group transfers, including endo- and exopeptidases (serine, cysteine, metallo and acid proteases); amino group and glutamyl transfers, including glutaminases, y glutamyl transpeptidases, amidotransferases, etc.; phosphoryl group transfers, including phosphotases, phosphodiesterases, kinases, and phosphorylases; nucleotidyl and pyrophosphotyl transfers, including carboxylate, pyrophosphoryl transfers, etc.; glycosyl group transfers; enzymes that do enzymatic oxidation and reduction, such as dehydrogenases, monooxygenases, oxidases, hydroxylases, reductases, etc.; enzymes that catalyze eliminations, isomerizations and rearrangements, such as elimination/addition of water using aconitase, fumarase, enolase, crotonase, carbon-nitrogen lyases, etc.; and enzymes that make or break carbon-carbon bonds, i.e. carbanion reactions. Suitable enzymes are listed in the Swiss-Prot enzyme database.

Thus, in one embodiment, the methods herein can be employed to screen for changes in the expression or state of enzymatic activity of specific proteins. These changes may be induced by a variety of chemicals, including pharmaceutical agonists or antagonists, or potentially harmful or toxic materials. The knowledge of such changes may be useful for diagnosing enzyme-based diseases and for investigating complex regulatory networks in cells.

Suitable viruses as sources of analytes to be labeled include, but are not limited to, orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella□zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like) Suitable bacteria include, but are not limited to, Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like.

In addition, any number of different cell types or cell lines may be evaluated using the labeling molecules of the invention.

Particularly preferred are disease state cell types, including, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cell lines, including, but not limited to, Jurkat T cells, NIH3T3 cells, CHO, Cos, etc.

The methods herein can also be used to implement a variety of clinical and diagnostic analyses to detect the presence, absence, deficiency or excess of a given protein or protein function in a biological fluid (e.g., blood), or in cells or tissue. The method is particularly useful in the analysis of complex mixtures of proteins, i.e., those containing 5 or more distinct proteins or protein functions.

In one embodiment, the cells may be genetically engineered, that is, contain exogeneous nucleic acid, for example, when the effect of additional genes or regulatory sequences on expressed proteins is to be evaluated.

In some embodiments, the target analyte may not be a protein; that is, in some instances, as will be appreciated by those in the art, other cellular components, including carbohydrates, lipids, nucleic acids, etc., can be labeled as well. In general this is done using the same or similar types of chemistry except that the reactive groups may be different.

The event of contacting the target protein with a dye reagent of the invention is referred to as a labeling reaction. During the incubation step the reactive group of the dye reagent forms a covalent bond with the target analyte or reference analyte present in the samples. The covalent bond is formed between the reactive group and the analyte under conditions well known in the art, See Example 12

As is known in the art, conditions that may affect the efficiency of the labeling reaction include the sensitivity of labeling reaction to pH, buffer type, and the salts in the reaction medium. In one embodiment of the invention, the labeling reaction is performed near pH 8.5. Amine-containing buffers are generally avoided to prevent potential cross-reactions with the amine reactive functional linker groups when such groups are used. Preferred buffers include, but are not limited to, phosphate, phosphate/borate, tertiary amine buffers such as BICINE, and borate. Additional agents that may be added to the labeling reaction included various detergents, urea, and thiourea.

Examples of reactive groups that form covalent bonds with amine groups of proteins are imidoesters and N-hydroxysuccinimidyl esters, sulfosuccinimidyl esters, isothiocyanates, aldehydes, sulfonylchlorides, or arylating agents. Amine groups are present in several amino acids, including lysine. Lysine ε-amino groups are very common in proteins (typically 6-7/100 of the residues) and the vast majority of the lysines are located on protein surfaces, where typically they are accessible to labeling.

In another embodiment of the invention, thiol groups of the target protein are used as the reactive group attachment site. Examples of reactive groups that form covalent bonds with thiol groups are sulfhydryl-reactive maleimides, iodoacetamides, alkyl bromides, or benzoxidiazoles.

The efficiency and progress of the labeling reaction, also referred to as labeling kinetics, can be measured by quenching the labeling reaction at different times with excess glycine, hydroxyl amine or other amine. The number of dyes per labeled protein and the relative fluorescence of the dyes on different labeled proteins can be determined using methods well known to those of skill in the art. For example, the number of optical labeling molecules per labeled protein and the relative fluorescence of the optical labeling molecules on different labeled proteins can be determined by separating the labeled proteins from the free optical label, using HPLC gel filtration with in-line fluorescence and absorbance detection. The ratio of hydrolyzed and unreacted optical label can be determined on the free optical label fraction by RP-HPLC (reverse-phase HPLC), if desired to help optimize labeling conditions. Isolated, labeled proteins can be incubated and run again on gel filtration to determine the stability of protein-dye reagent labeled proteins (Miyairi S., et al., (1998) Anal Biochem. 258(2):168-75; Mills J S, et al. (1998), J Biol. Chem. 273(17):10428-35; Kwon G, et al., (1993), Biochemistry, 32(9):2401-8).

In one aspect of the invention the present dye reagents are utilized in a proteomics experiment that typically involves the analysis of the proteins present in a cellular extract of the intact organism, tissue, cell or subcellular fraction before and after exposure to a particular physiological stimulus. In one embodiment, proteins that are present in the extract of the cells prior to exposure to the physiological stimuli are labeled with one of the dye reagents. Proteins that are present in the extract of the cells after exposure to the physiological stimuli are labeled with a matched dye reagent that is distinguished from the first dye reagent by mass, after different strengths of physiological stimuli are applied. The dye labeled proteins from two or more cellular extracts are mixed and then simultaneously separated and analyzed by observing the optical signals of the separated proteins, thus permitting the identification of the proteins which are detectably altered in expression level or post-translational modification state in response to the stimuli of interest and facilitating a further focused study of these proteins and their post-translational modifications. In one embodiment of the invention, the presence or absence of the labeled proteins is analyzed to determine if a specific protein is affected by the presence or absence of the physiological stimuli. In a further embodiment of the invention, the relative quantity (or ratios of expression) of the specific labeled proteins is determined.

In a preferred embodiment, the plurality of matched dye labeled proteins are separated prior to determining the ratios of expression or post-translational modification of the matched dye labeled proteins. The labeled proteins may be separated using, for example, 1D gel electrophoresis, 2D gel electrophoresis, capillary electrophoresis, 1D chromatography, 2D chromatography, 3D chromatography, liquid chromatography (LC) or mass spectroscopy (MS). In a preferred embodiment of the invention, the large number of labeled proteins are separated by LC and the proteins are analyzed by MS. In one aspect analysis includes sequencing of the peptide or protein wherein sequencing of the peptide identifies the protein the peptide originated from. In another aspect, analysis includes measuring the amount of the labeled protein or peptide in the sample, this step typically includes the addition of a known amount of one or more internal standards for each of the proteins or peptides to be quantitated.

Internal standards, which are appropriately isotopically labeled, may be employed in the methods of this invention to measure absolute quantitative amounts of proteins in samples. Internal standards are of particular use in assays intended to quantitate dye reagent labeled products of enzymatic reactions. In this application, the internal standard is chemically identical to the labeled enzymatic product generated by the action of the enzyme on the dye reagent labeled enzyme substrate, but carries isotope labels which may include ²H, ¹³C, ¹⁵N, ¹⁷O, ¹⁸O, or ³⁴S, that allow it to be independently detected by MS techniques. Internal standards for use in the method herein to quantitative one or several proteins in a sample are prepared by reaction of dye reagent with a known protein to generate the dye reagent labeled peptides generated from digestion of the labeled protein. Dye reagent labeled peptide internal standards are substantially chemically identical to the corresponding target dye labeled peptides generated from digestion of dye reagent labeled protein, except that they are differentially isotopically labeled to allow their independent detection by MS techniques.

Thus, the method of this invention can be applied to determine the relative quantities of one or more proteins in two or more protein samples, the proteins in each sample are reacted with dye reagents which are substantially chemically identical but differentially isotopically labeled. The samples are combined and processed as one. The relative quantity of each labeled peptide which reflects the relative quantity of the protein from which the peptide originates is determined by the measurement of the respective isotope peaks by mass spectrometry.

In a further aspect of the invention, the various post-translational modifications are identified. Post-translational modifications include phosphorylation, methionine oxidation, cysteine oxidation to sulfenic acid, tyrosine nitration, thiol nitrosylation, disulfide formation, glycoslyation, carboxylation, acylation, methylation, sulfation, and prenylation.

A further aspect of the invention provides for methods of determining whether a particular protein is exposed to the surface of its native environment. In one embodiment of the invention, a first dye reagent is used to label exposed target proteins on the surfaces of cells, isolated organelles or isolated multiprotein complexes. The cell or organelle membranes or the multiprotein complex structure are then disrupted with detergents and the interior groups labeled with a second matched dye reagent. The sample is then separated by a method described above, preferably by LC. Those proteins labeled with the first dye reagent are proteins exposed to the surface of the cell, organelle or multiprotein complex. Those proteins labeled with the second dye reagent molecule are proteins that are not exposed to the surface of cell, organelle or multiprotein complex. In a preferred embodiment of the invention, the labeled proteins are isolated and identified, as described above.

In various aspects, the invention is drawn to mass spectroscopy. As used herein, the term “mass spectrometry” (or simply “MS”) encompasses any spectrometric technique or process in which molecules are ionized and separated and/or analyzed based on their respective molecular weights. Thus, as used herein, the terms “mass spectrometry” and “MS” encompass any type of ionization method, including without limitation electrospray ionization (ESI), atmospheric-pressure chemical ionization (APCI) and other forms of atmospheric pressure ionization (API), and laser irradiation. Mass spectrometers are commonly combined with separation methods such as gas chromatography (GC) and liquid chromatography (LC). GC or LC separates the components in a mixture, and the components are then individually introduced into the mass spectrometer; such techniques are generally called GC/MS and LC/MS, respectively. MS/MS is an analogous technique where the first-stage separation device is another mass spectrometer. In LC/MS/MS, the separation methods comprise liquid chromatography and MS. Any combination (e.g., GC/MS/MS, GC/LC/MS, GC/LC/MS/MS, etc.) of methods can be used to practice the invention. In such combinations, “MS” can refer to any form of mass spectrometry; by way of non-limiting example, “LC/MS” encompasses LC/ESI MS and LC/MALDI-TOF MS. Thus, as used herein, the terms “mass spectrometry” and “MS” include without limitation APCI MS; ESI MS; GC MS; MALDI-TOF MS; LC/MS combinations; LC/MS/MS combinations; MS/MS combinations; etc.

It is often necessary to prepare samples comprising an analyte of interest for MS. Such preparations include without limitation purification and/or buffer exchange. Any appropriate method, or combination of methods, can be used to prepare samples for MS. One preferred type of MS preparative method is liquid chromatography (LC), including without limitation HPLC and RP-HPLC.

High-pressure liquid chromatography (HPLC) is a separative and quantitative analytical tool that is generally robust, reliable and flexible. Reverse-phase (RP) is a commonly used stationary phase that is characterized by alkyl chains of specific length immobilized to a silica bead support. RP-HPLC is suitable for the separation and analysis of various types of compounds including without limitation biomolecules, (e.g., glycoconjugates, proteins, peptides, and nucleic acids, and, with mobile phase supplements, oligonucleotides). One of the most important reasons that RP-HPLC has been the technique of choice amongst all HPLC techniques is its compatibility with electrospray ionization (ESI). During ESI, liquid samples can be introduced into a mass spectrometer by a process that creates multiple charged ions (Wilm et al., Anal. Chem. 68:1, 1996). However, multiple ions can result in complex spectra and reduced sensitivity.

In HPLC, peptides and proteins are injected into a column, typically silica based C18. An aqueous buffer is used to elute the salts, while the peptides and proteins are eluted with a mixture of aqueous solvent (water) and organic solvent (acetonitrile, methanol, propanol). The aqueous phase is generally HPLC grade water with 0.1% acid and the organic solvent phase is generally an HPLC grade acetonitrile or methanol with 0.1% acid. The acid is used to improve the chromatographic peak shape and to provide a source of protons in reverse phase LC/MS. The acids most commonly used are formic acid, trifluoroacetic acid, and acetic acid. In RP HPLC, compounds are separated based on their hydrophobic character. With an LC system coupled to the mass spectrometer through an ESI source and the ability to perform data-dependant scanning, it is now possible in at least some instances to distinguish proteins in complex mixtures containing more than 50 components without first purifying each protein to homogeneity. Where the complexity of the mixture is extreme, it is possible to couple ion exchange chromatography and RP-HPLC in tandem to identify proteins from mixtures containing in excess of 1,000 proteins.

A particular type of MS technique, matrix-assisted laser desorption time-of-flight mass spectrometry (MALDI-TOF MS) (Karas et al., Int. J. Mass Spectrom. Ion Processes 78:53, 1987), has received prominence in analysis of biological polymers for its desirable characteristics, such as relative ease of sample preparation, predominance of singly charged ions in mass spectra, sensitivity and high speed. MALDI-TOF MS is a technique in which a UV-light absorbing matrix and a molecule of interest (analyte) are mixed and co-precipitated, thus forming analyte:matrix crystals. The crystals are irradiated by a nanosecond laser pulse. Most of the laser energy is absorbed by the matrix, which prevents unwanted fragmentation of the biomolecule. Nevertheless, matrix molecules transfer their energy to analyte molecules, causing them to vaporize and ionize. The ionized molecules are accelerated in an electric field and enter the flight tube. During their flight in this tube, different molecules are separated according to their mass to charge (m/z) ratio and reach the detector at different times. Each molecule yields a distinct signal. The method is used for detection and characterization of biomolecules, such as proteins, peptides, oligosaccharides and oligonucleotides, with molecular masses between about 400 and about 500,000 Da, or higher. MALDI-MS is a sensitive technique that allows the detection of low (10⁻¹⁵ to 10⁻¹⁸ mole) quantities of analyte in a sample.

Partial amino acid sequences of proteins can be determined by enzymatic proteolysis followed by MS analysis of the product peptides. These amino acid sequences can be used for in silico examination of DNA and/or protein sequence databases. Matched amino acid sequences can indicate proteins, domains and/or motifs having a known function and/or tertiary structure. For example, amino acid sequences from an uncharacterized protein might match the sequence or structure of a domain or motif that binds a ligand. As another example, the amino acid sequences can be used in vitro as antigens to generate antibodies to the protein and other related proteins from other biological source material (e.g., from a different tissue or organ, or from another species). There are many additional uses for MS, particularly MALDI-TOF MS, in the fields of genomics, proteomics and drug discovery. For a general review of the use of MALDI-TOF MS in proteomics and genomics, see Bonk et al. (Neuroscientist 7:12, 2001).

Tryptic peptides labeled with light or heavy dye reagents can be directly analyzed using MALDI-TOF. However, where sample complexity is apparent, on-line or off-line LC-MS/MS or two-dimensional LC-MS/MS is necessary to separate the peptides. For example, for simple digests, a gradient of 5-45% (v/v) acetonitrile in 0.1% formic acid (or TFA, if MALDI MS/MS is available) over 45 min, and then 45-95% acetonitrile in 0.1% formic acid (or TFA, if MALDI MS/MS is available) over 5 min can be used. 0.1% Formic acid solution is used on the Q-TOF instrument and 0.1% TFA solution is used on the Dionex Probot fraction collector for off-line coupling between HPLC and MALDI-MS/MS analysis (carried out on the ABI 4700). For a complex sample, a gradient of 5-45% (v/v) acetonitrile over 90 min, and then 45-95% acetonitrile over 30 min is used. For a very complex sample, a gradient of 5-45% (v/v) acetonitrile over 120 min, and then 45-95% acetonitrile over 60 min might be used. On the Q-TOF, one survey scan and four MS/MS data channels are used to acquire CID data with 1.4 s scan time. On the 4700 proteomics, the most intense eight peptides with mass over 1000 are chosen for MS/MS analysis.

The foregoing methods having been described it is understood that the many and varied compounds of the present invention can be utilized with the many methods. The compounds not being limited to just those that are specifically disclosed. Compounds that include stable isotopes in the dye moiety, linker or both can be utilized. Compounds that optionally comprise a second reactive group or an affinity tag can also be utilized.

### KITS

Suitable kits for binding, detecting and identifying analytes in a differentially labeled sample also form part of the invention. Such kits can be prepared from readily available materials and reagents and can come in a variety of embodiments. The contents of the kit will depend on the design of the assay protocol or reagent for detection or measurement. All kits will contain instructions, a present compound and appropriate reagents, as needed. Typically, instructions include a tangible expression describing the reagent concentration or at least one assay method parameter such as the relative amounts of reagent and sample to be added together, maintenance time periods for reagent/sample admixtures, temperature, buffer conditions and the like to allow the user to carry out any one of the methods or preparations described above.

Therefore, kits of the present invention comprise at least one compound of the present invention in an appropriate storage form, e.g. lyophilized or dissolved in an organic solvent, and instructions for preparing the compounds to be used by the researcher. In addition, the kits may contain appropriate controls (including a positive control), calibration standards, buffer solutions and additional detection reagents such as dye-conjugates, or a reference dye standard.

In one aspect, a kit comprises a first dye reagent comprising at least one stable isotope wherein the reagent has the formula D-L-R: wherein D is a dye moiety, L is a linker and R is a reactive group that selectively reacts with a functional group of a protein; and a second dye reagent that is substantially chemically identical to the first dye reagent but isotopically distinguishable.

A detailed description of the invention having been provided above, the following examples are given for the purpose of illustrating the invention and shall not be construed as being a limitation on the scope of the invention or claims.

### EXAMPLES

### Example 1

### Synthesis of Compound 4

Benzoic-ring-¹³C₆ acid (1) is nitrated by reaction with excess nitric acid. The carboxylic acid moiety directs nitration to the m-position to give arene 2 (¹³C carbon atoms are denoted by asterisks). The carboxylic acid in 2 is reduced to the alcohol with excess borane in hot THF, followed by oxidation to the aldehyde by reaction with excess pyridinium chlorochromate (PCC) in dichloromethane; reaction of the resulting aldehyde to the formate ester 3 is accomplished by reaction with excess 3-chloroperbenzoic acid (MCPBA) in dichloromethane. The nitro group in 3 is reduced to an amino group by catalytic hydrogenation, followed by bis-methylation with excess dimethylsulfate in DMF mediated by diisopropylethylamine (DIEA); the formate ester is cleaved with excess aqueous KOH in methanol to give ring-¹³C₆-3-dimethylaminophenol (4).

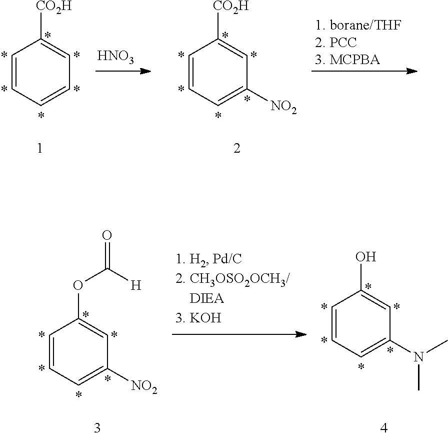

### Example 2

### Synthesis of Compound 7

A solution of two equivalents of 4 is condensed with trimellitic anhydride (5) in warm propionic acid with catalytic p-toluenesulfonic acid (TSA), followed by HPLC-based separation of regioisomers to give rhodamine 6 which contains twelve ¹³C atoms at the asterisk-indicated positions. Rhodamine 6 is converted into the amine reactive ester 7 by reaction with excess disuccinimidyl carbonate in the presence of catalytic 4-dimethylaminopyridine (DMAP).

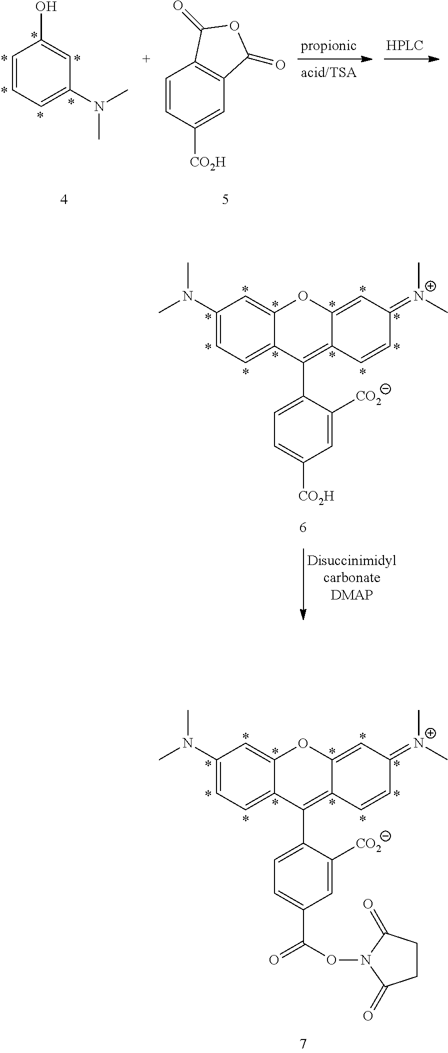

### Example 3

### Synthesis of Compound 10

A solution of two equivalents of 4 is condensed with 4-nitrophthalic anhydride (8) in warm sulfuric acid, followed by HPLC-based separation of regioisomers to give rhodamine 9 that contains twelve ¹³C atoms at the asterisk-indicated positions. The nitro group in 9 is reduced to an amino group with excess sodium sulfide ion methanol and water, and the amino group is converted into a thiol-reactive iodoacetamide moiety by reaction with two equivalents of iodoacetic anhydride in chloroform to give rhodamine 10.

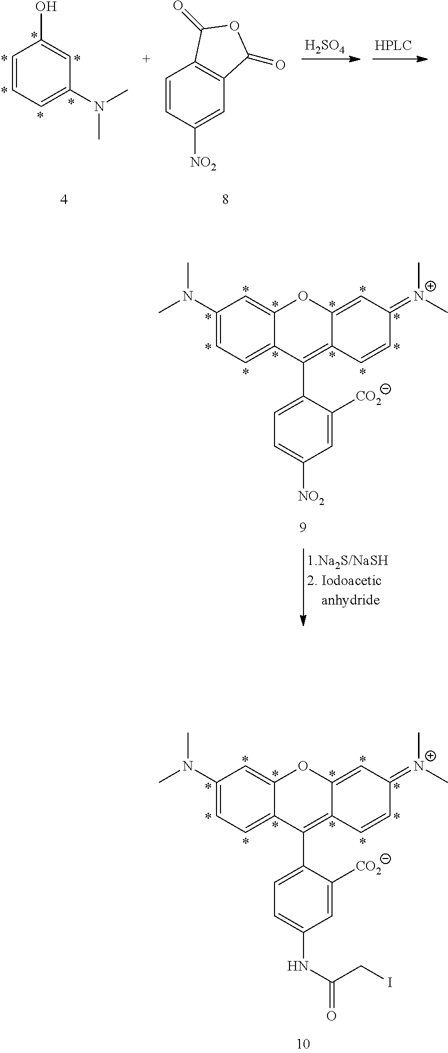

### Example 4

### Synthesis of Compound 11

The ¹³C-substituted rhodamine succinimidyl ester 7 is reacted with excess cadaverine (1,5-diaminopentane), followed by acylation of the resulting primary amine with excess N-(t-BOC-aminooxyacetic acid, tetrafluorophenyl ester, followed by deprotection of the resulting carbamate with trifluoroacetic acid (TFA) to give aldehyde-reactive hydroxylamine 11.

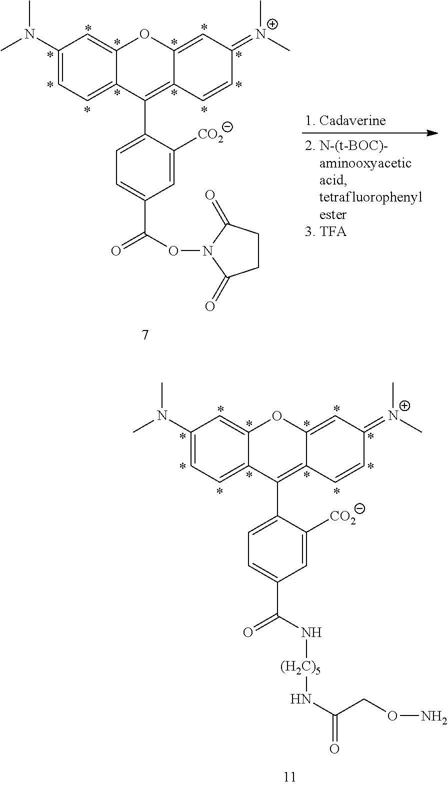

### Example 5

### Synthesis of Compound 14

A hot melt of two equivalents of ¹³C-substituted 4 with one equivalent of 2-sulfobenzoic acid anhydride (12) gives the sulforhodamine 13 containing ¹³C atoms (twelve) at the asterisked positions. The sulfonic acid is converted into a sulfonyl chloride by reaction with excess phosphorous oxychloride (POCl₃), followed by treatment with excess piperazine in THF to give a mono-sulfonamide, followed by treatment with excess iodoacetic anhydride in chloroform to give the thiol-reactive iodoacetamide 14.

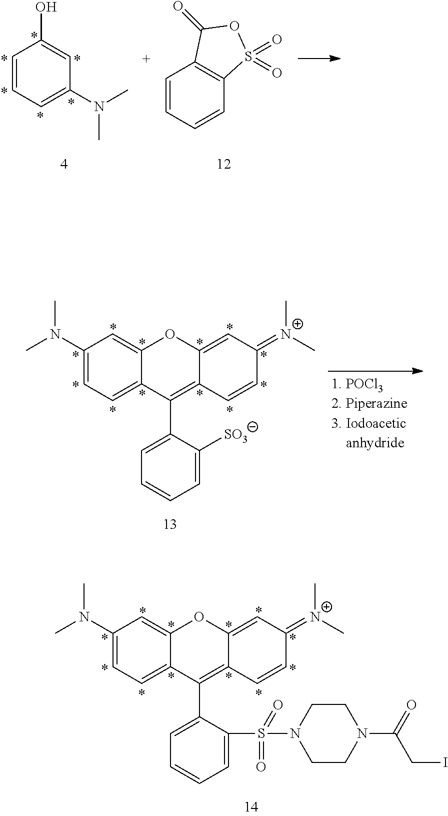

### Example 6

### Synthesis of Compounds 17 and 18

Benzoic-ring-¹³C₆ acid (1) is esterified with methanol and catalytic sulfuric acid, followed by conversion into the corresponding dimethylphthalate with carbon dioxide and diazomethane according to the chemistry described in J. Am. Chem. Soc. 1989, 111(20): 8016-8; treatment with excess aqueous potassium hydroxide to cleave the methyl esters, followed by acidification with aqueous hydrochloric acid (HCl) gives the ring-¹³C₆-phthalic acid 15. Condensation of two equivalents of 4-fluororesorcinol with 15 in warm methanesulfonic acid gives ring-¹³C₆-substituted fluorescein 16 after aqueous workup. A Mannich reaction of 16 with 0.8 eq of N-hydroxymethylchloroacetamide in cool sulfuric acid, followed by treatment with hot aqueous hydrochloric acid, followed by reaction of the resulting primary amine with excess iodoacetic anhydride in DMF gives the thiol-reactive 17 with ¹³C atoms at the asterisked positions. The carboxylic acid moiety in 17 is condensed with one equivalent of ethylenediamine-biotin, mediated by one equivalent of dicyclohexylcarbodiimide (DCC) in acetonitrile to give affinity labeled probe 18.

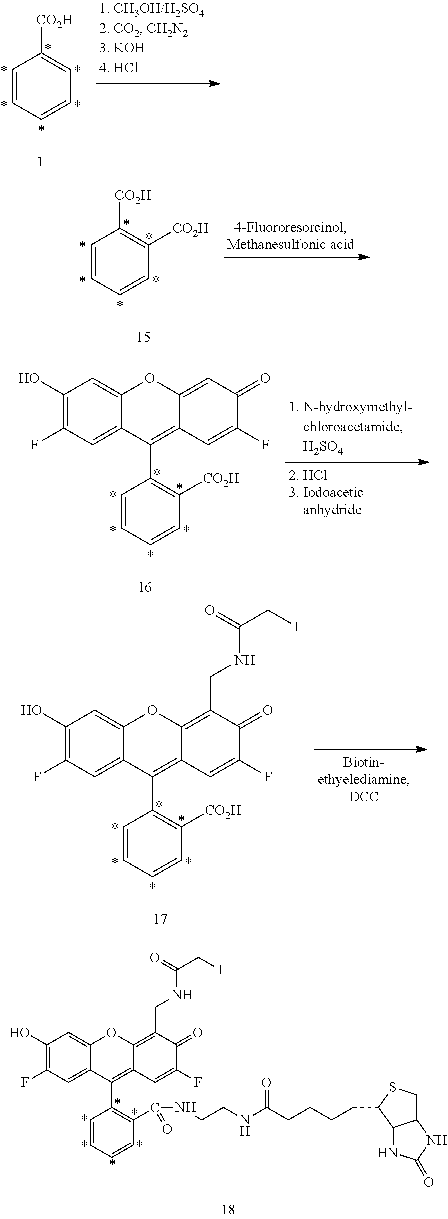

### Example 7

### Synthesis of Compounds 22, 23, and 24

Reaction of the known rhodamine sulfonyl chloride 19 with excess fully ¹³C and ¹⁵N labeled proline (20, Sigma-Aldrich) in DMF and pyridine gives carboxylic acid 21 in which the asterisked carbon and nitrogen atoms are substituted with ¹³C and ¹⁵N, respectively. Reaction of 21 with excess disuccinimdyl carbonate and catalytic DMAP in THF gives amine-reactive succinimidyl ester 22, which contains six stable heavy isotopes in the linker. Acylation of the amino group in biocytin with 22, followed by conversion of the resulting carboxylic acid with excess disuccinimidyl carbonate and catalytic DMAP in THF gives the amine-reactive affinity tag-containing dye 23, which contains six stable heavy isotopes in the linker. Also, reaction of 22 with excess ethylenediamine in THF, followed by reaction of the resulting primary amine with excess iodoacetic anhydride in THF, gives thiol-reactive dye 24 that contains six stable heavy isotopes in the linker.

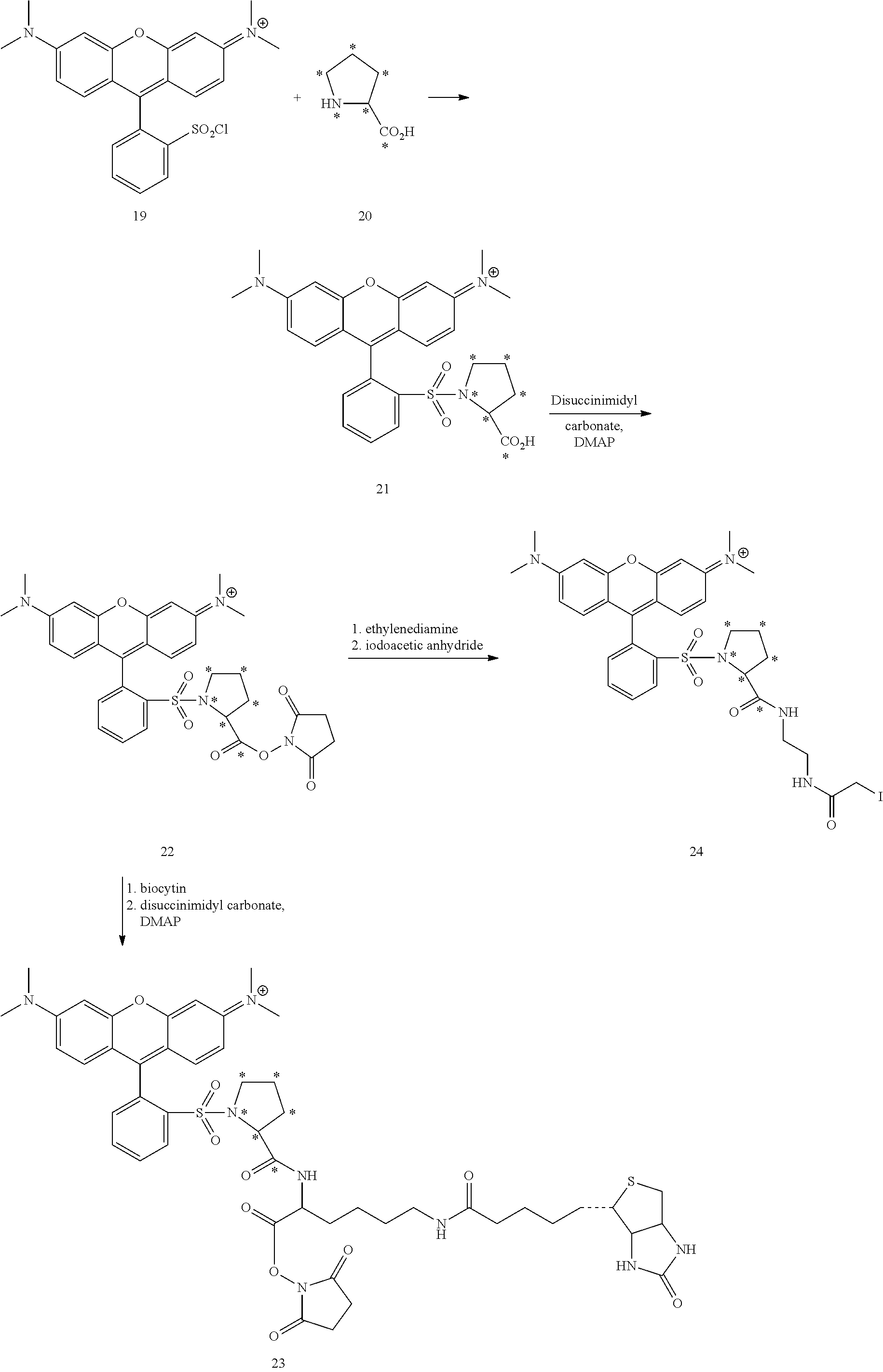

### Example 8

### Synthesis of Compound 27

Reaction of the known sulfonyl chloride 19 with excess 2,2,3,3,5,5,6,6-octadeuterio-piperazine (prepared from d₄-ethylenediamine and alcoholic ammonia) in DMF and pyridine gives sulfonamide 26, which is reacted with excess iodoacetic anhydride in chloroform to give thiol-reactive dye 27, which contains eight stable heavy isotopes (deuteriums) in the linker.

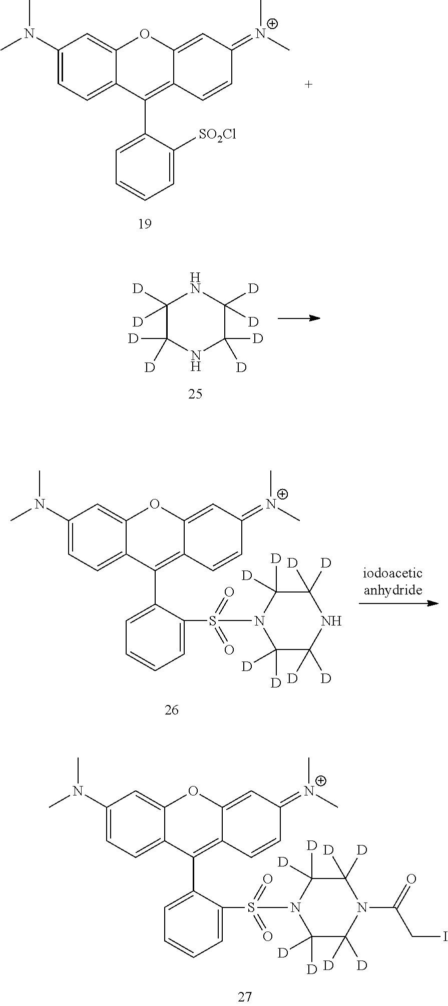

### Example 9

### Synthesis of Compounds 32 and 33

Similar to the preparation of compound 4, benzoic-d₅ acid (28, Aldrich) is nitrated by reaction with excess deuterio-nitric acid. The carboxylic acid moiety directs nitration to the m-position to give arene 29. The carboxylic acid in 29 is reduced to the alcohol with excess borane in hot THF, followed by oxidation to the aldehyde by reaction with excess pyridinium chlorochromate (PCC) in dichloromethane; reaction of the resulting aldehyde to the formate ester 30 is accomplished by reaction with excess 3-chloroperbenzoic acid (MCPBA) in dichloromethane. The nitro group in 30 is reduced to an amino group by catalytic hydrogenation, followed by bis-methylation with excess dimethylsulfate in DMF mediated by diisopropylethylamine (DIEA); the formate ester is cleaved with excess aqueous KOH in methanol to give d₄-dimethylaminophenol (31). Two equivalents of 31 are condensed with one equivalent of 2-sulfobenzoic acid cyclic anhydride as a hot melt, followed by reaction of the resulting sulfonic acid with phosphorous oxychloride and pyridine gives the amine-reactive sulfonyl chloride 32 which contains six heavy isotopes (deuterium) attached to the dye moiety. Reaction of 32 with excess piperazine in DMF, followed by reaction of the resulting secondary amine with excess iodoacetic anhydride in chloroform, gives thiol-reactive iodoacetamide 33 which contains six heavy isotopes (deuterium) attached to the dye moiety.

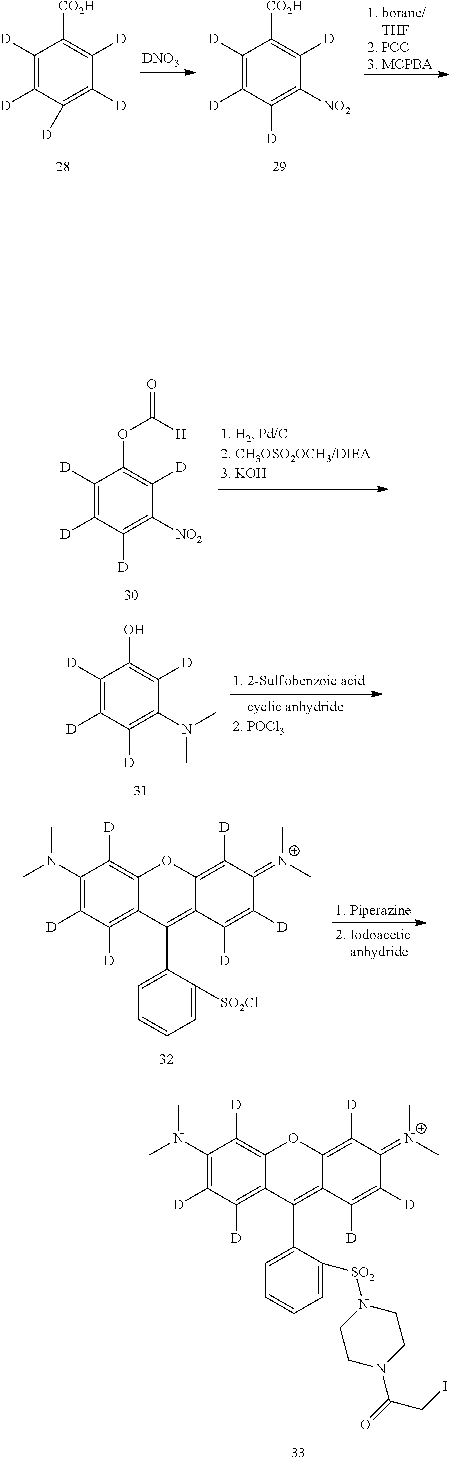

### Example 10

### Synthesis of Compound 36

Similar to the preparation of Compound 15, benzoic-d₅ acid (28, Aldrich) is esterified with excess d₁-methanol using catalytic deuterio-sulfuric acid, followed by conversion into the corresponding dimethylphthalate with carbon dioxide and diazomethane according to the chemistry described in J. Am. Chem. Soc. 1989, 111(20): 8016-8; treatment with excess aqueous potassium hydroxide to cleave the methyl esters, followed by acidification with deuterio-hydrochloric acid (DCl) in D₂O gives the d₄-phthalic acid 34. Condensation of 34 with two equivalents of 4-fluororesorcinol in warm methanesulfonic acid gives the tetradeuterated fluorescein 35 after aqueous workup. A Mannich reaction of 35 with 0.8 eq of N-hydroxymethylchloroacetamide in cool sulfuric acid, followed by treatment with hot aqueous hydrochloric acid, followed by reaction of the resulting primary amine with excess iodoacetic anhydride in DMF gives the thiol-reactive 36 which contains four deuterium atoms attached to the dye moiety.

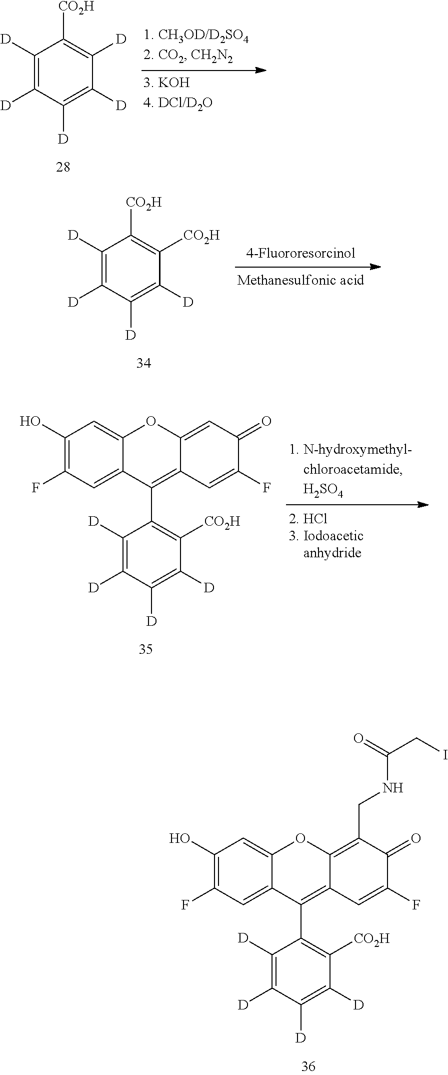

### Example 11

### Synthesis of Compound 37-42

### 6-TAMRA-L-Proline

To a solution of L-proline (5.4 mg, 0.047 mmol) in triethylammonium buffer (1.0 M, pH=8.5, 0.24 mL) was added 6-carboxytetramethyl rhodamine, succinimidyl ester (6-TAMRA-SE, 25.0 mg, 0.047 mmol). After stirring the reaction solution at RT for 1.5 h, the solution was concentrated to afford a residue. The residue was reevaporated from water two times and then dissolved in a small amount of water and passed through Dowex 50WX8-200 ion-exchange resin (4.0 cm×2.5 cm) to remove any trace L-proline. The product, which stuck to the column, was washed with water (50 mL) then eluted with concentrated ammonia hydroxide. All of the eluent containing product (as noted by the presence of the bright pink color) was concentrated to afford a purple solid (25 mg, 99%). TLC (8:1:1, CH₃CN:H₂O:AcOH)R_{f}=0.20.

### 6-TAMRA-L-Proline-SE

To a suspension of 6-TAMRA-L-proline (22 mg, 0.041 mmol) in CH₃CN (0.2 mL) and DIEA (18 μL, 0.1 mmol) was added O—(N-succinimidyl)-N,N,N′,N′-tetramethyluronium tetrafluoroborate (25 mg, 0.082 mmol). The solution, which became homogeneous upon addition of the tetrafluoroborate, was stirred at RT for 15 min, quenched with 1% AcOH (1.0 mL) and diluted with CHCl₃ (20 mL). The aqueous layer was extracted three times with CHCl₃ (3×20 mL) and the combined organics were dried over Na₂SO₄. Following filtration and concentration, the product was obtained as a purple solid (26 mg, 99%). TLC (DIWA: dioxane, 15 mL: isopropanol, 58 mL; H₂O, 13 mL; NH₄OH, 14 mL) quenching of the SE with 2-methoxyethylamine) R_{f}=0.68.

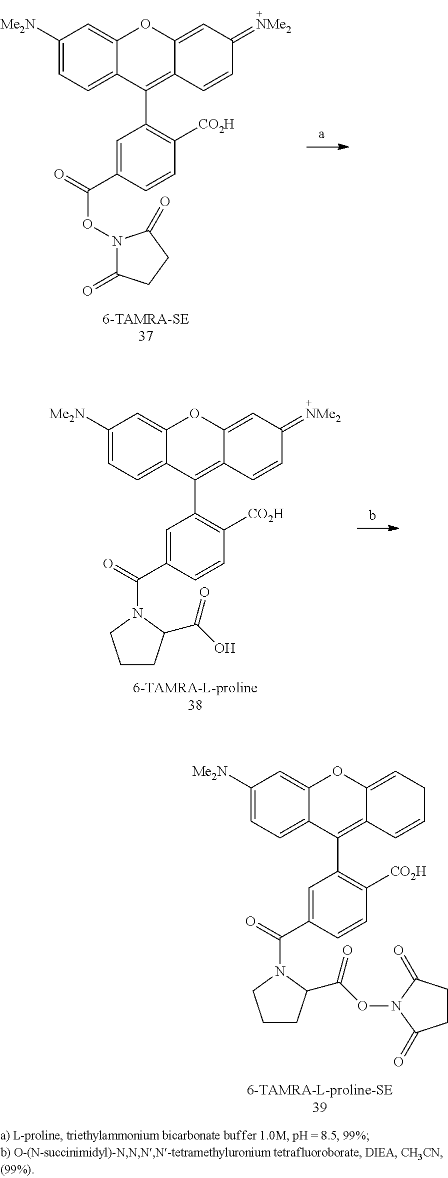

### “Heavy” 6-TAMRA-L-Proline

To a solution of L-proline (6.0 mg, 0.047 mmol, Cambridge Isotopes Lab, ¹³C5, ¹⁵N1) in triethylammonium buffer (1.0 M, pH=8.5, 0.24 mL) was added 6-carboxytetramethyl rhodamine, succinimidyl ester (6-TAMRA-SE, 25.0 mg, 0.047 mmol). After stirring the reaction solution at RT for 1.5 h, the solution was concentrated to afford a residue. The residue was reevaporated from water two times and then dissolved in a small amount of water and passed through Dowex 50WX8-200 ion-exchange resin (4.0 cm×2.5 cm) to remove any trace L-proline. The product, which stuck to the column, was washed with water (50 mL) then eluted with concentrated ammonia hydroxide. All of the eluent containing product (as noted by the presence of the bright pink color) was concentrated to afford a purple solid (25 mg, 99%). TLC (8:1:1, CH₃CN:H₂O:AcOH)R_{f}=0.20.

### “Heavy” 6-TAMRA-L-Proline-SE

To a suspension of heavy 6-TAMRA-L-proline (20 mg, 0.038 mmol) in CH₃CN (0.2 mL) and DIEA (16 μL, 0.1 mmol) was added O—(N-succinimidyl)-N,N, N′,N′-tetramethyluronium tetrafluoroborate (23 mg, 0.076 mmol). The solution, which became homogeneous upon addition of the tetrafluoroborate, was stirred at RT for 15 min, quenched with 1% AcOH (1.0 mL) and diluted with CHCl₃ (20 mL). The aqueous layer was extracted three times with CHCl₃ (3×20 mL) and the combined organics were dried over Na₂SO₄. Following filtration and concentration, the product was obtained as a purple solid (26 mg, 99%). TLC (DIWA: dioxane, 15 mL: isopropanol, 58 mL; H₂O, 13 mL; NH₄OH, 14 mL) quenching of the SE with 2-methoxyethylamine) R_{f}=0.68.

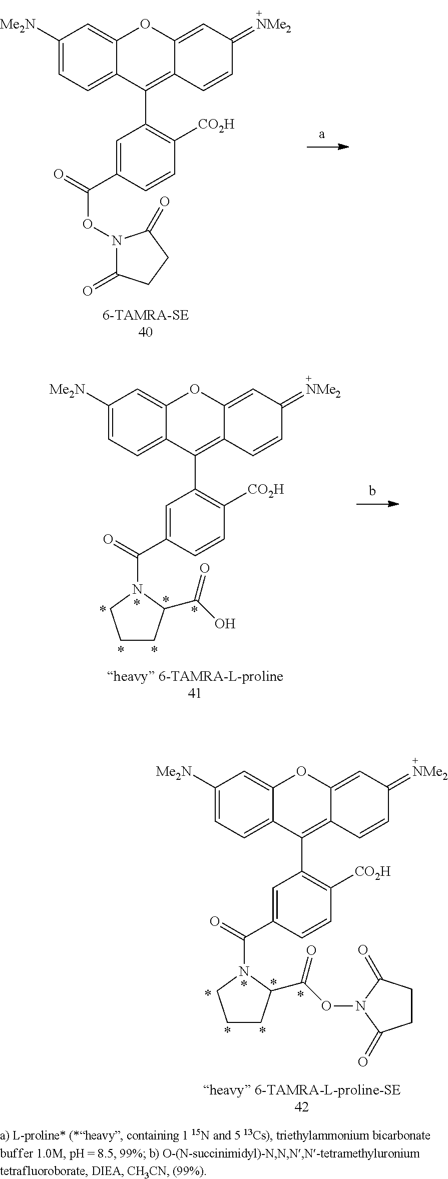

### Example 12

### Labeling of the AT1 Peptide with Heavy and Light 6-TAMRA-Proline Succinimidyl Ester Compounds (Compound 39 and 42) (Mass Difference=6 Daltons)

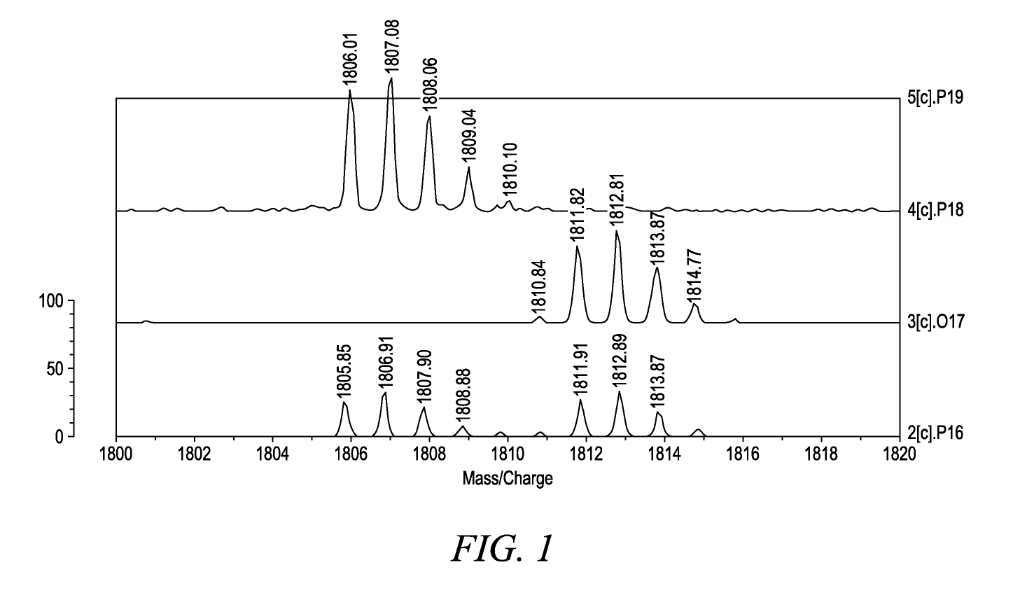

Five μL of 5 mg/mL Angiotensin I (Sigma A-9650 (DRVYIHPFHL, M+1=1296.88), 5 mg/mL (3.9 μmol/μL)), 5 uL of 40 nmol/μL TAMRA-SE (heavy or light reagents, Compound 39 (MW 631.65, 40 nmol/μL solution prepared by solubilizing 5 mg in 200 μL DMF) or 42 (MW 625.65, 40 nmol/μL solution prepared by solubilizing 5 mg in 200 μL DMF)), 25 μL of 200 mM sodium bicarbonate pH 9, and 15 μL e-pure water were combined in a microcentrifuge tube and incubated under argon at ambient temperature with gentle vortexing for 2 hours. A 1:1 mixture of the heavy and light labeling mixture was prepared and the commixture was diluted 100-fold in 0.1% TFA. 0.5 μL spots were applied to the MALDI plate, and these were mixed with 0.5 μL of 10 mg/mL α-cyano-4-hydroxycinnamic acid in 50% acetonitrile/0.1% TFA by pipeting up and down. Then, the samples were air dried. Samples were analyzed by MALDI in positive reflectron mode. FIG. 1. shows the MALDI analysis results of the co-mixture of equal amounts of heavy and light labeled AT1 (lower panel), as well as, the individual heavy and light labeled reagents (upper panels). The lower panel, as expected, shows two species differing by a mass weight of 6 amu at the expected mass weights of 1806 and 1812 consistent with the addition of compound 39 or 42 with encoded linker.

We claim:

1. A method for identifying and determining the relative amounts of one or more proteins in two or more samples, wherein the method comprises the steps:
a) contacting each sample with a dye reagent that is substantially chemically identical but isotopically distinguishable to provide contacted samples, wherein said dye reagent has the formula:
D-L-R
wherein D is a dye moiety, L is a linker and R is a reactive group that selectively reacts with a functional group of a protein wherein either the dye moiety or the linker or both are labeled with one or more stable isotopes;
b) incubating each sample with one of the isotopically distinguishable dye reagents to provide discrete sets of dye reagent labeled proteins, dye reagent labeled proteins in different samples being thereby differently labeled with one or more stable isotopes;
c) combining the discrete sets of differentially labeled samples to provide a pooled labeled sample; and,
d) detecting, measuring and determining the pooled differentially labeled proteins whereby the relative amounts of proteins are identified and determined.

2. The method according to claim 1, wherein said dye reagent further comprises an affinity reagent.

3. The method according to claim 2, wherein the affinity reagent is selected from the group consisting of a hapten, glutathione, a metal chelating moiety, protein A, protein G and maltose.

4. The method according to claim 1, wherein the dye reagent labeled proteins are digested or fragmented to convert to dye labeled peptides.

5. The method according to claim 1, wherein the method further comprises separating differentially labeled proteins and/or peptides from proteins and/or peptides that are not labeled with the dye reagent.

6. The method according to claim 1, wherein the detecting comprises optically visualizing the differentially labeled proteins and/or peptides.

7. The method according to claim 1, wherein the reactive group is carboxylic acid, succinimidyl ester of a carboxylic acid, hydrazide, amine, tetrafluorophenyl ester, isothiocyanate, sulfonyl chloride, a photoactivatable group or a maleimide.

8. The method according to claim 1, wherein the dye moiety is xanthene, borapolyazaindacene, cyanine, coumarin, acridine, furan, indole, quinoline, benzofuran, quinazolinone, or benzazole.

9. The method according to claim 8, wherein the xanthene is fluorescein, rhodamine, rosamine, rhodol or derivatives thereof.

10. The method according to claim 1, wherein the dye moiety comprises one or more stable isotopes.

11. The method according to claim 1, wherein the stable isotopes are independently ²H, ¹³C, ¹⁵N, ¹⁷O, ¹⁸O, ¹⁸F or ³⁴S.

12. The method according to claim 1, wherein the linker is a single covalent bond or a covalent linkage that is linear or branched, cyclic or heterocyclic, saturated or unsaturated, having 1-20 nonhydrogen atoms selected from the group consisting of C, N, P, O and S; and are composed of any combination of ether, thioether, amine, ester, carboxamide, sulfonamide, hydrazide bonds and aromatic or heteroaromatic bonds.

13. The method according to claim 1, wherein the linker contains a cleavable moiety.

14. The method according to claim 1, wherein the linker comprises one or more stable isotopes.

15. The method according to claim 1, wherein the dye reagent tagged protein is digested or fragmented to convert them to dye reagent tagged peptides.

16. The method according to claim 15, wherein said peptides are sequenced by mass spectrometry.

17. A dye reagent, wherein the dye reagent has the formula:
D-L-R
wherein D is a dye moiety, L is a linker and R is a reactive group that selectively reacts with a functional group of a protein wherein the dye moiety or linker contains at least one stable isotope.

18. The dye reagent according to claim 17, wherein the dye reagent further comprises an affinity reagent.

19. The dye reagent according to claim 18, wherein the affinity reagent is selected from the group consisting of a hapten, glutathione, a metal chelating moiety, protein A, protein G and maltose.

20. The dye reagent according to claim 17, wherein the dye reagent further comprises a second reactive group.

21. The dye reagent according to claim 17, wherein the dye moiety is xanthene, borapolyazaindacene, cyanine, coumarin, acridine, furan, indole, quinoline, benzofuran, quinazolinone, or benzazole.

22. The dye regent according to claim 21, wherein the xanthene is fluorescein, rhodamine, rosamine, rhodol or derivatives thereof.

23. The dye reagent according to claim 17, wherein the dye moiety comprises one or more stable isotopes.

24. The dye reagent according to claim 17, wherein the stable isotopes are 2H, ¹³C, ¹⁵N, ¹⁷O, ¹⁸O, ¹⁸F or ³⁴S.

25. The dye reagent according to claim 17, wherein the reactive group is carboxylic acid, succinimidyl ester of a carboxylic acid, hydrazide, amine, tetrafluorophenyl ester, isothiocyanate, sulfonyl chloride, photoactivatable group or a maleimide.

26. The dye reagent according to claim 17, wherein the linker is a single covalent bond or a covalent linkage that is linear or branched, cyclic or heterocyclic, saturated or unsaturated, having 1-20 nonhydrogen atoms selected from the group consisting of C, N, P, O and S; and are composed of any combination of ether, thioether, amine, ester, carboxamide, sulfonamide, hydrazide bonds and aromatic or heteroaromatic bonds.

27. The dye reagent according to claim 17, wherein the linker contains a cleavable moiety.

28. The dye reagent according to claim 17, wherein the linker comprises one or more stable isotopes.

29. A kit for labeling protein or peptides in a sample, wherein the kit comprises:
a) a first dye reagent comprising at least one stable isotope wherein the reagent has the formula D-L-R:
wherein D is a dye moiety, L is a linker and R is a reactive group that selectively reacts with a functional group of a protein; and
b) a second dye reagent that is substantially chemically identical to the first dye reagent but isotopically distinguishable.
